(19) United States
(12) Patent Application Publication
(10) Pub. No.: US 2025/0304586 A1
(43) Pub. Date: Oct. 2, 2025

(54) CRYSTALLINE POLYMORPHS OF N-METHYL-N-((1S,3S)-3-METHYL-3-((6-(1-METHYL-1H-PYRAZOL-4-YL)PYRAZOLO[1,5-A]PYRAZIN-4-YL)OXY)CYCLOBUTYL)ACRYLAMIDE

(71) Applicant: BIOGEN MA INC., Cambridge, MA (US)

(72) Inventors: Fengmei Zheng, Quincy, MA (US); Bo Wang, Arlington, MA (US); Yiqing Lin, Lexington, MA (US)

(21) Appl. No.: 18/864,351

(22) PCT Filed: May 9, 2023
(86) PCT No.: PCT/US2023/021522

§ 371 Date: Nov. 8, 2024

Related U.S. Application Data
(60) Provisional application No. 63/340,348, filed on May 10, 2022.

(51) Int. Cl. C07D 487/04

(52) U.S. Cl. CPC C07D 487/04

(57) ABSTRACT

Provided are crystalline forms of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide, pharmaceutical compositions, methods of use and methods of making thereof.

### RELATED APPLICATIONS

The present application claims the benefit of U.S. Provisional Application Ser. No. 63/340,348 filed on May 10, 2022, the contents of which are incorporated by reference herein.

### TECHNICAL FIELD

The present disclosure relates to novel crystalline polymorphs of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide. These polymorphs can be used for treating a disorder responsive to inhibition of Bruton's tyrosine kinase. In another aspect, the disclosure relates to a process for preparation of the novel polymorphs.

### BACKGROUND

Protein kinases are a large multigene family consisting of more than 500 proteins which play a critical role in the development and treatment of a number of human diseases in oncology, neurology and immunology. The Tec kinases are non-receptor tyrosine kinases which consists of five members (Tec (tyrosine kinase expressed in hepatocellular carcinoma), Btk (Bruton's tyrosine kinase), Itk (interleukin-2 (IL-2)-inducible T-cell kinase; also known as Emt or Tsk), Rlk (resting lymphocyte kinase; also known as Txk) and Bmx (bone-marrow tyrosine kinase gene on chromosome X; also known as Etk)) and are primarily expressed in haematopoietic cells, although expression of Bmx and Tec has been detected in endothelial and liver cells. Tec kinases (Itk, Rlk and Tec) are expressed in T cell and are all activated downstream of the T-cell receptor (TCR). Btk is a downstream mediator of B cell receptor (BCR) signaling which is involved in regulating B cell activation, proliferation, and differentiation. More specifically, Btk contains a PH domain that binds phosphatidylinositol (3,4,5)-trisphosphate (PIP3). PIP3 binding induces Btk to phosphorylate phospholipase C (PLCy), which in turn hydrolyzes PIP2 to produce two secondary messengers, inositol triphosphate (IP3) and diacylglycerol (DAG), which activate protein kinase PKC, which then induces additional B-cell signaling. Mutations that disable Btk enzymatic activity result in XLA syndrome (X-linked agammaglobulinemia), a primary immunodeficiency. Given the critical roles which Tec kinases play in both B-cell and T-cell signaling, Tec kinases are targets of interest for autoimmune disorders.

Consequently, there is a great need in the art for effective inhibitors of Btk.

### SUMMARY

The present disclosure relates to crystalline forms (or polymorphs) of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide (compound 1) or a salt thereof. In certain embodiments, the crystalline forms of the present application have improved stability and suitability for pharmaceutical uses. Other advantages may include favorable pharmacokinetic properties, ease of isolation, process reproducibility, suitability for large scale manufacturing process, etc.

In one embodiment, the present disclosure provides crystalline Form A of compound 1.

In another embodiment, the present disclosure provides crystalline Form B of compound 1.

In another embodiment, the present disclosure provides crystalline Form I of maleate salt of compound 1.

In another embodiment, the present disclosure provides crystalline Form II of tartrate salt of compound 1.

In yet another embodiment, the present disclosure provides crystalline Form III of tartrate salt of compound 1.

In another embodiment, the present disclosure provides crystalline Form IV of citrate salt of compound 1.

In another embodiment, the present disclosure provides crystalline Form V of proline salt of compound 1.

The present disclosure also provides a pharmaceutical composition comprising at least one polymorph described herein and at least one pharmaceutically acceptable excipient.

One aspect of the present disclosure provides a method of treating a disorder responsive to inhibition of Btk in a subject comprising administering to said subject an effective amount of a composition (e.g., a pharmaceutical composition) comprising a polymorph described herein.

The present disclosure also includes the use of a composition (e.g., a pharmaceutical composition) comprising a polymorph described herein for the manufacture of a medicament for the treatment of a disorder responsive to inhibition of Btk. Also provided is a polymorph described herein for use in treating a disorder responsive to inhibition of Btk.

### BRIEF DESCRIPTION OF THE FIGURES

FIG. 1 depicts a powder X-ray diffraction (PXRD) pattern of crystalline Form A of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide.

FIG. 2 is a single crystal structural representation for the asymmetric unit cell structure of crystalline Form A of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide.

FIG. 3 depicts differential scanning calorimetry (DSC) and thermal gravimetric analysis (TGA) profiles of crystalline Form A of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide.

FIG. 4 shows ¹H NMR spectrum of crystalline Form A of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide.

FIG. 5 depicts a powder X-ray diffraction (PXRD) pattern of crystalline Form B of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide.

FIG. 6 is a single crystal structural representation for the asymmetric unit cell structure of crystalline Form B of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide.

FIG. 7 depicts differential scanning calorimetry (DSC) and thermal gravimetric analysis (TGA) profiles of crystalline Form B of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide.

FIG. 8 shows ¹H NMR spectrum of crystalline Form B of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide.

FIG. 9 depicts a powder X-ray diffraction (PXRD) pattern of crystalline Form I of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide maleate salt.

FIG. 10 depicts differential scanning calorimetry (DSC) and thermal gravimetric analysis (TGA) profiles of crystalline Form I of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide maleate salt.

FIG. 11 shows ¹H NMR spectrum of crystalline Form I of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide maleate salt.

FIG. 12 depicts a powder X-ray diffraction (PXRD) pattern of crystalline Form II of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide tartrate salt.

FIG. 13 depicts differential scanning calorimetry (DSC) and thermal gravimetric analysis (TGA) profiles of crystalline Form II of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide tartrate salt.

FIG. 14 shows ¹H NMR spectrum of crystalline Form II of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide tartrate salt.

FIG. 15 depicts a powder X-ray diffraction (PXRD) pattern of crystalline Form III of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide tartrate salt.

FIG. 16 depicts differential scanning calorimetry (DSC) and thermal gravimetric analysis (TGA) profiles of crystalline Form III of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide tartrate salt.

FIG. 17 shows ¹H NMR spectrum of crystalline Form III of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide tartrate salt.

FIG. 18 depicts a powder X-ray diffraction (PXRD) pattern of crystalline Form IV of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide citrate salt.

FIG. 19 depicts differential scanning calorimetry (DSC) and thermal gravimetric analysis (TGA) profiles of crystalline Form IV of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide citrate salt.

FIG. 20 shows ¹H NMR spectrum of crystalline Form IV of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide citrate salt.

FIG. 21 depicts a powder X-ray diffraction (PXRD) pattern of crystalline Form V of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide proline salt.

FIG. 22 depicts differential scanning calorimetry (DSC) and thermal gravimetric analysis (TGA) profiles of crystalline Form V of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide proline salt.

FIG. 23. shows ¹H NMR spectrum of crystalline Form V of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide proline salt.

### DETAILED DESCRIPTION

As used herein, the term “compound” refers to N-methyl-N-((1s,3s)-3-methyl-3-((6-(1-methyl-1H-pyrazol-4-yl)pyrazolo[1,5-a]pyrazin-4-yl)oxy)cyclobutyl)acrylamide. The structure for the compound is shown below:

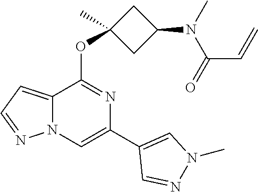 [SMILES: C=CC(=O)N(C)[C@H]1C[C@@](C)(Oc2nc(-c3cnn(C)c3)cn3nccc23)C1]

As used herein, the term “crystalline”, “crystalline form” or “polymorph” refers to a solid form having a crystal form wherein the individual molecules have a highly homogeneous regular locked-in chemical configuration. The crystalline form can be characterized by analytical methods, such as powder X-ray diffraction (PXRD), differential scanning calorimetry (DSC), thermogravimetric analysis (TGA), NMR, etc.

As used herein, an “anti-solvent crystallization” method involves the addition of an anti-solvent to a solution comprising the compound, which drastically reduces the solubility of the compound and results in the precipitation or crystallization of the compound. The precipitation of the compound can occur immediately or slowly over time. In some embodiments, after the addition of the anti-solvent, the resulting mixture can be cooled to a low temperature (e.g., below room temperature, between 0° C. and 10° C., or between 0° C. and 5° C.) to facilitate the precipitation of the crystalline form. Thereafter, the precipitate (crystals) may easily be separated by filtration, decanting, or centrifugation.

The term “anti-solvent”, as used herein, refers to a solvent in which the compound is insoluble or has very low solubility. Suitable anti-solvents include, but are not limited to, water, hydrocarbons, including petroleum ether, pentane, hexane(s), heptane, octane, isooctane, cyclopentane, cyclohexane, methylcyclohexane, benzene, toluene, xylene, n-butanol.

As used herein, a “reverse anti-solvent crystallization” method involves the addition of a solution of the compound (obtained by dissolving the compound in a solvent to form a clear solution) to an anti-solvent until a precipitate appears. Alternatively, the solution is added to added to a fixed volume of the anti-solvent. The desired crystalline form can form or precipitate out slowly over time. In some embodiments, after the addition of the solvent, the resulting mixture can be cooled to a low temperature (e.g., below room temperature, between 0° C. and 10° C., or between 0° C. and 5° C.) to facilitate the precipitation or crystallization of the crystalline form. Thereafter, the precipitate (crystals) may easily be separated by filtration, decanting, or centrifugation.

As used herein “slurry cycling crystallization” method comprises suspending the compound in a solvent followed by heating and slow cooling, wherein the heating and cooling steps can be optionally repeated for 1-10 times to yield the desired crystalline form. The mixture of the compound and the solvent can be heated to a temperature between 30° C. and 150° C., between 30° C. and 100° C., between 30° C. and 70° C., or between 40° C. and 60° C. In one embodiment, the mixture can be heated to 50° C. The mixture can be heated at the desired temperature for a period of time, e.g., between 10 minutes and 10 hours, between 10 minutes and 5 hours, between 10 minutes and 2 hours, between 10 minutes and 1 hour, between 20 minutes and 40 minutes, or between 1 hours and 5 hours. In one embodiment, the mixture is heated for 30 minutes. The heated mixture can then be cooled down slowly to room temperature or a low temperature between 0° C. and 15° C., or between 0° C. and 10° C. or between 0° C. and 5° C. In one embodiment, the mixture can be cooled to 5° C. The cooling is carried out slowly, for example, at a rate of 0.1-0.5° C./minutes (e.g., 0.1° C./minute).

As used herein, “slurry conversion crystallization” method involves stirring of the suspension of the compound in a solvent for a period time sufficient for the conversion of the compound from one solid form to another solid form. In some embodiments, the mixture of the compound and the solvent can be stirred for 1-5 hours, for 1-10 hours, for 1 hour to 1 day, for 1 day to 10 days, or for 1 day to 5 days. In some embodiments, the mixture is stirred for 1 day, 2 days, 3 days, 4 days or 5 days.

As used herein, a “slurry” method includes “slurry cycling crystallization” and “slurry conversion crystallization”. In some embodiments, a slurry method is “slurry conversion crystallization”.

As used herein “liquid vapor diffusion crystallization” method involves the diffusion of the vapor of a volatile solvent, in which the compound is not soluble or has low solubility, into a solution containing the compound. The vapor of the volatile solvent diffuses into the solution, decreasing the overall solubility of the compound and resulting in the compound to precipitate out of the solution. In some embodiments, the method is carried out by adding the volatile solvent to the solution and keeping the resulting mixture in a sealed container. In some embodiments, the solution can be evaporated to dryness at room temperature.

As used herein “ionic liquid induced crystallization” method involves dissolving the compound in a solvent in the presence of an ionic liquid, and allowing the slow evaporation of the solvent to yield the desired solid form of the compound. Exemplary ionic liquid include, but are not limited to, 1,3-dimethylimidazolium trifluoroacetic acid ([dmim]CF₃COOH), 1,3-dimethylimidazolium perchlorate ([dmim]ClO₄), 1-butyl-3-methylimidazolium hexafluorophosphate ([bmim]PF₆) and 1-ethyl-3-methylimidazolium hexafluroantimonate ([emim]SbF₆).

As used herein, “polymer induced crystallization” method involves stirring a solution of compound in a solvent in the presence of a polymer mixture to yield the desired solid form. Exemplary polymer mixture include, but are not limited to, a mixture of polymers selected from polyvinyl pyrrolidone (PVP), polyvinyl alcohol (PVA), polyvinylchloride (PVC), polyvinyl acetate (PVAC), hypromellose (HPMC), and methyl cellulose (MC). In some embodiments, the polymer mixture is a mixture of polyvinyl pyrrolidone (PVP), polyvinyl alcohol (PVA), polyvinylchloride (PVC), polyvinyl acetate (PVAC), hypromellose (HPMC), and methyl cellulose (MC) with mass ratio of 1:1:1:1:1:1).

As used herein, “fast evaporation crystallization” method involves dissolving a solid form of the compound in a solvent followed by fast evaporation of the solvent to yield the desired crystalline form. Fast solvent evaporation can be achieved, for example, by exposing the solution of the compound to air at room temperature to allow the volatile solvent to evaporate. Alternatively, the solvent can be evaporated under vacuum and/or at an elevated temperature (e.g., higher than room temperature).

The term “crystalline Form A” “crystalline Form B”, “crystalline Form I”, “crystalline Form II”, “crystalline Form III”, “crystalline Form IV” or “crystalline Form V” relates to specific crystalline form of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide or a specific salt thereof as defined below.

The present disclosure relates to various crystalline polymorphs of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide or a specific salt thereof and a process for preparing the same. Any suitable crystallization method known in the art can be used to prepared the crystalline forms of the compound or a salt thereof described herein. Exemplary crystallization methods include, but are not limited to, anti-solvent crystallization method, reverse anti-solvent crystallization method, slurry cycling crystallization method, slurry conversion crystallization method, liquid vapor diffusion crystallization method, polymer induced crystallization method, and fast evaporation crystallization method.

In one aspect, the present disclosure provides crystalline Form A of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide (compound 1).

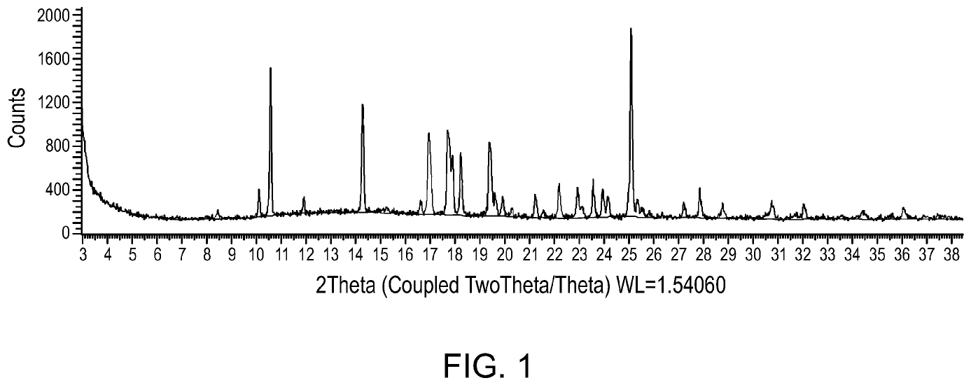

In one embodiment, crystalline Form A is characterized by at least three, at least four, at least five, at least six or at least seven powder X-ray diffraction (PXRD) peaks at 2θ angles selected from 10.1°, 10.6°, 14.3°, 16.9°, 17.8°, 18.2°, 19.4° and 25.1°. In another embodiment, crystalline Form A is characterized by PXRD peaks at 2θ angles of 10.1°, 10.6°, 14.3°, 16.9°, 17.8°, 18.2°, 19.4° and 25.1°. In another embodiment, crystalline Form A is characterized by at least three, at least four, at least five, at least six, at least seven, at least eight, at least nine, at least ten, at least eleven, at least twelve, or at least thirteen PXRD peaks at 2θ angles selected from 10.1°, 10.6°, 11.9°, 14.3°, 16.9°, 17.8°, 18.2°, 19.4°, 21.3°, 22.2°, 23.0°, 24.0°, 25.10 and 27.8°. In yet another embodiment, crystalline Form A is characterized by PXRD peaks at 2θ angles selected from 10.1°, 10.6°, 11.9°, 14.3°, 16.9°, 17.8°, 18.2°, 19.4°, 21.3°, 22.2°, 23.0°, 24.0°, 25.1° and 27.8°. In some embodiments, the peaks described in the above embodiments for crystalline Form A have a relative intensity of at least 1%, at least 2%, at least 5%, at least 10%, or at least 15%. In some embodiments, crystalline Form A has a PXRD pattern that is substantially the same as PXRD pattern shown in FIG. 1.

As used herein, the term “relative intensity” refers to a ratio of the peak intensity for the peak of interest versus the peak intensity for the largest peak.

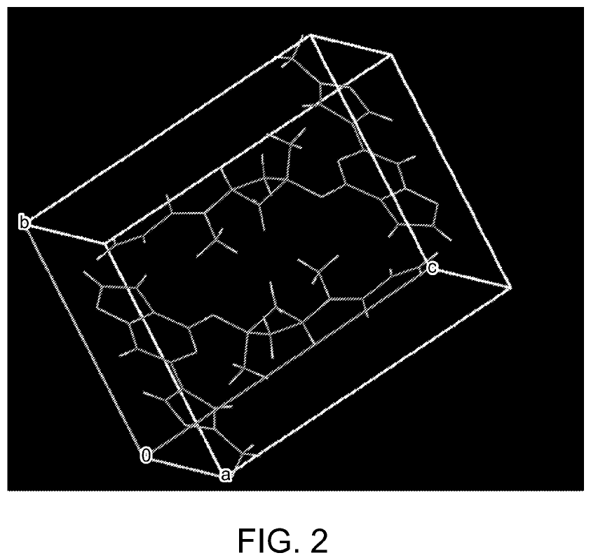

In some embodiments, crystalline Form A is characterized by single crystal X-ray crystallographic data obtained from suitable single crystals of Form A of compound 1 using Cu Kα radiation. The crystal structure is characterized as a P-1 space group. In a related embodiment of the disclosure, crystalline Form A of compound 1 is characterized by an assymetric unit cell structure with parameters listed in Table 1B. In one embodiment, the asymmetric unit cell has a volume of 904.872 Å3 and 3-D parameters of a=5.98064 Å; b=10.28273 Å; c=14.91842 Å. The unit cell is also characterized by Mercury drawing shown in FIG. 2. The unit cell consists of two N-methyl-N-((1s,3s)-3-methyl-3-((6-(1-methyl-1H-pyrazol-4-yl)pyrazolo[1,5-a]pyrazin-4-yl)oxy)cyclobutyl)acrylamide molecules.

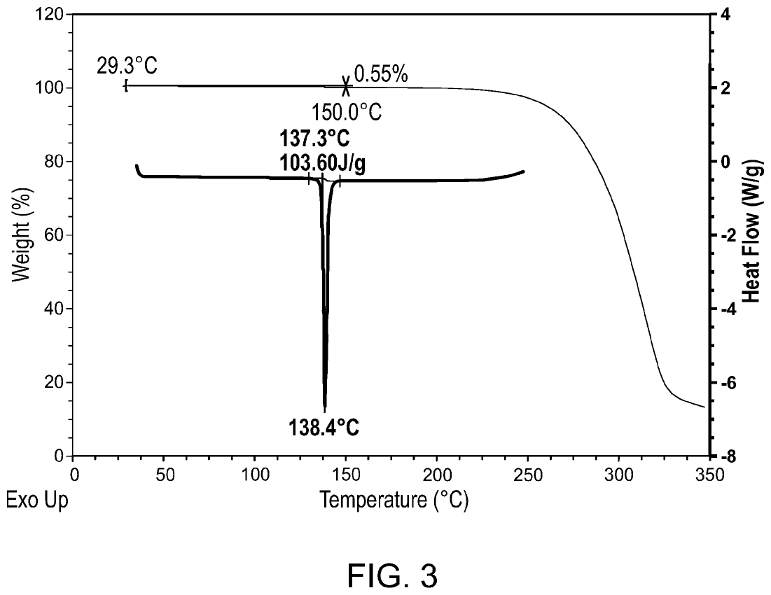

In some embodiments, crystalline Form A has a DSC profile that is substantially the same as DSC profile shown in FIG. 3. In particular, crystalline Form A is characterized by an onset temperature at 137.3° C.±2° C. in the DSC profile. In another embodiment, crystalline Form A has a melting temperature of 138.4° C.±2° C.

In some embodiments, crystalline Form A has a TGA profile that is substantially the same as the TGA profile shown in FIG. 3. In particular, the TGA profile indicates that crystalline Form A is a non-hygroscopic anhydrate.

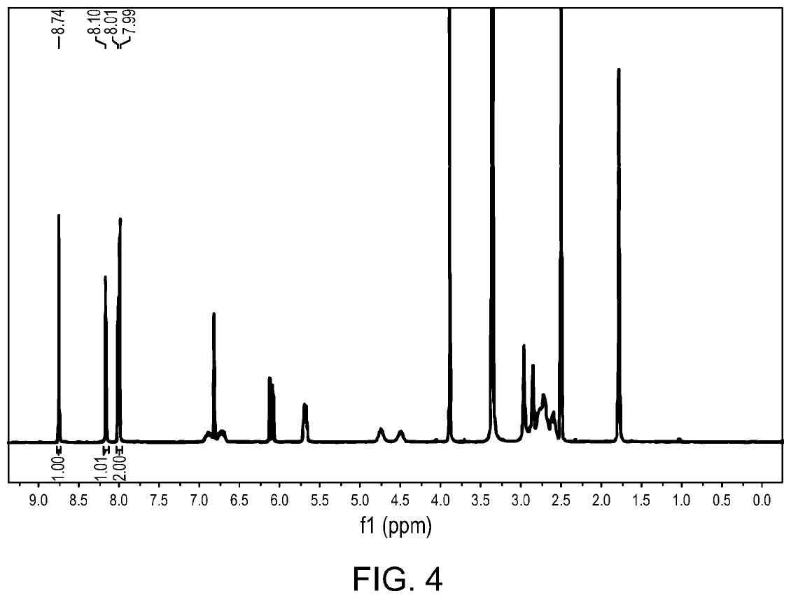

In another embodiment, crystalline Form A is characterized by the ¹H NMR as shown in FIG. 4.

In some embodiments, crystalline Form A is characterized by, for example, ¹H NMR DSC, TGA and PXRD. In one embodiment, crystalline Form A is characterized by PXRD alone or PXRD in combination with one or more of DSC, TGA and ¹H NMR described above.

“Non-hygroscopic” as used herein, means that the crystalline form cannot readily absorb or adsorb water from its surroundings.

“Anhydrate” or “anhydrous” as used herein, means that the crystalline form comprises substantially no water in the crystal lattice e.g., less than 1% by weight as determined by, for example, TGA analysis or other quantitative analysis.

In some embodiments, crystalline Form A is at least 70%, 80%, 85%, 90%, 95%, 97%, 99%, 99.5% or 99.9% pure. The purity of Form A is determined by dividing the weight of crystalline Form A in a composition comprising compound 1 over the total weight of the compound in the composition. In one embodiment, the present disclosure provides a composition comprising compound 1, wherein at least 70%, 80%, 85%, 90%, 95%, 97%, 99%, 99.5% or 99.9% by weight of the compound in the composition is crystalline Form A of the compound.

In one aspect, the present disclosure provides a method for preparing crystalline Form A of compound 1. In one embodiment, the method is a slurry method described herein. In a particular embodiment of the above disclosed method, crystalline Form A can be obtained using crystalline Form B as starting material, isopropyl alcohol (IPA) as the solvent. In one embodiment, crystalline Form A can be obtained by slurrying (stirring) crystalline Form B in IPA at room temperature (RT) for a period time (e.g., for 1 hour to 8 hours, for 1 hour to 4 hours, for one day to one week, for 2 or more weeks, for 1, 2, 3, 4, 5, 6 or 7 days, etc.) that is sufficient to form Form A. In some embodiment, crystalline Form A can be obtained by slurrying crystalline Form B in IPA at room temperature (RT) for 6 days.

In another aspect, the present disclosure provides crystalline Form B of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide.

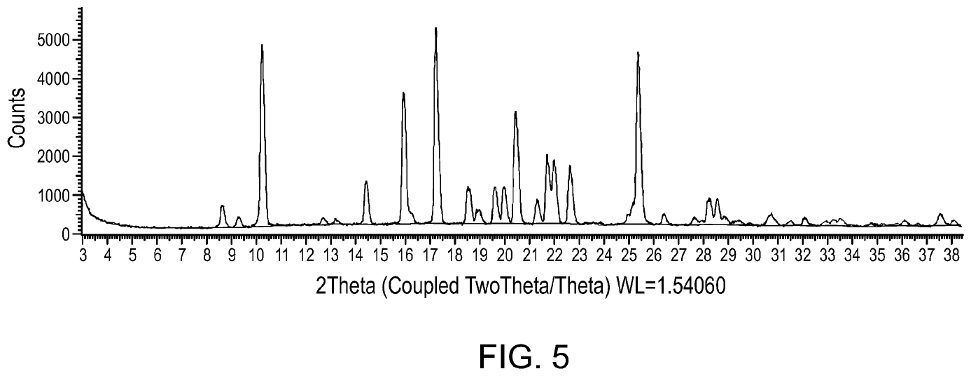

In one embodiment, crystalline Form B is characterized by at least three, at least four, at least five, at least six or at least seven powder X-ray diffraction (PXRD) peaks at 2θ angles selected from 8.6°, 9.3°, 10.2°, 14.4°, 16.0°, 17.3°, 20.4°, and 25.4°. In another embodiment, crystalline Form B is characterized by PXRD peaks at 2θ angles of 8.6°, 9.3°, 10.2°, 14.4°, 16.0°, 17.3°, 20.4°, and 25.4°. In another embodiment, crystalline Form B is characterized by at least three, at least four, at least five, at least six, at least seven, at least eight, at least nine, at least ten, at least eleven, or at least twelve PXRD peaks at 2θ angles selected from 8.6°, 9.3°, 10.2°, 14.4°, 16.0°, 17.3°, 18.7°, 20.4°, 21.4°, 21.9°, 22.7°, 25.4° and 28.4°. In yet another embodiment, crystalline Form B is characterized by PXRD peaks at 2θ angles selected from 8.6°, 9.3° 10.2°, 14.4°, 16.0°, 17.3°, 18.7°, 20.4°, 21.4°, 21.9°, 22.7°, 25.4° and 28.4°. In some embodiments, the peaks described in the above embodiments for crystalline Form B have a relative intensity of at least 1%, at least 2%, at least 5%, at least 10%, or at least 15%. In some embodiments, crystalline Form B has a PXRD pattern that is substantially the same as PXRD pattern shown in FIG. 5.

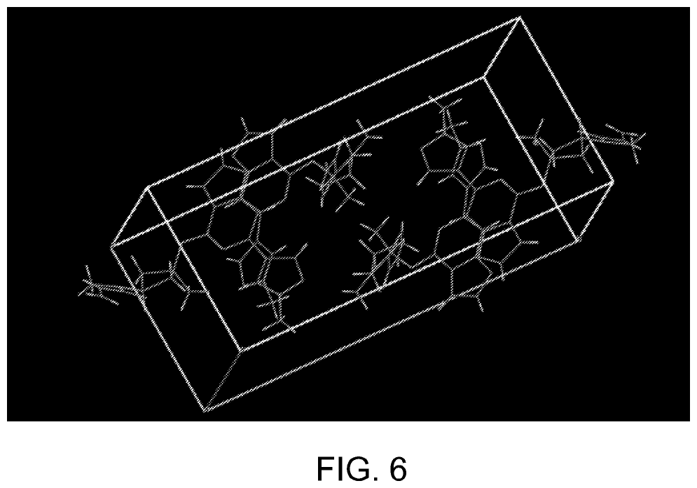

In some embodiments, crystalline Form B is characterized by single crystal X-ray crystallographic data obtained from suitable single crystals of Form B of compound 1 using CuKα radiation. The crystal structure is characterized as a P21/c space group. In a related embodiment of the disclosure, crystalline Form B of compound 1 is characterized by an assymetric unit cell structure with parameters listed in Table 2B. In one embodiment, the asymmetric unit cell has a volume of 1859.45 Å3 and 3-D parameters of a=9.4633 Å; b=20.6615 Å; c=9.5408 Å. The unit cell is also characterized by Mercury drawing shown in FIG. 6. The unit cell consists of four N-methyl-N-((1s,3s)-3-methyl-3-((6-(1-methyl-1H-pyrazol-4-yl)pyrazolo[1,5-a]pyrazin-4-yl)oxy)cyclobutyl)acrylamide molecules.

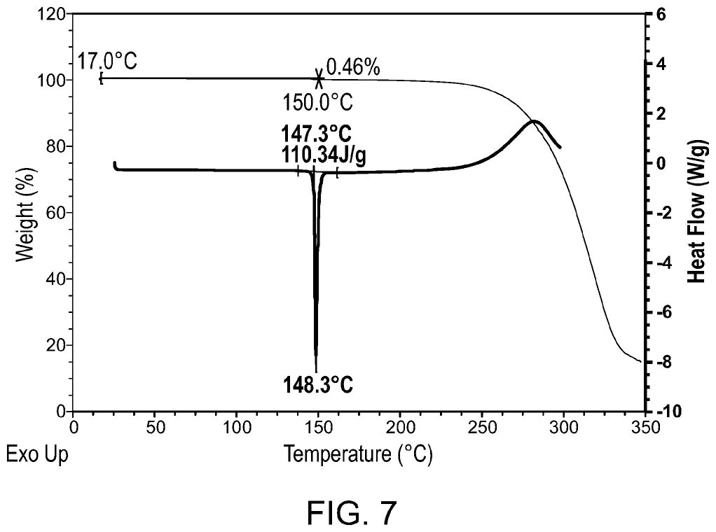

In some embodiments, crystalline Form B has a DSC profile that is substantially the same as DSC profile shown in FIG. 7. In particular, crystalline Form B is characterized by an onset temperature at 147.3° C.±2° C. in the DSC profile. In another embodiment, crystalline Form B has a melting temperature of 148.3° C.±2° C.

In some embodiments, crystalline Form B has a TGA profile that is substantially the same as the TGA profile shown in FIG. 7. In particular, the TGA profile indicates that crystalline Form B is a non-hygroscopic anhydrate.

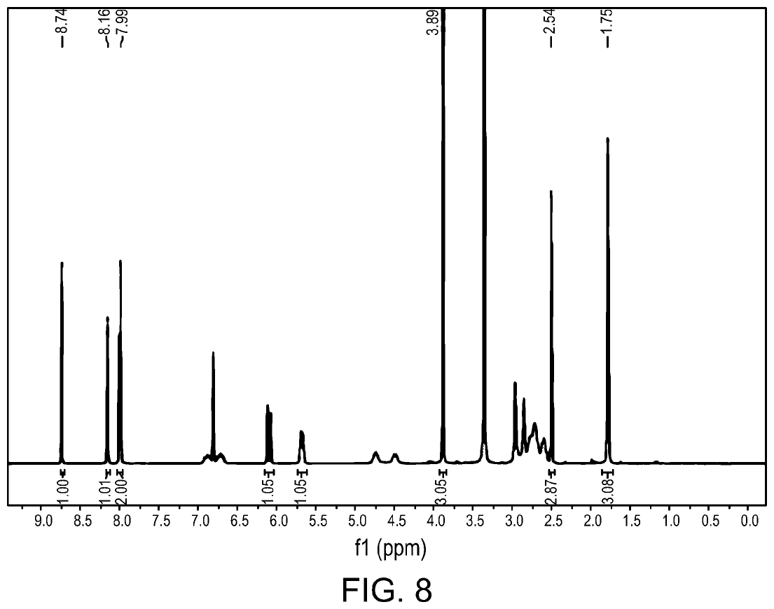

In another embodiment, crystalline Form B is characterized by the ¹H NMR as shown in FIG. 8.

In some embodiments, crystalline Form B is characterized by, for example, ¹H NMR DSC, TGA and PXRD. In one embodiment, crystalline Form B is characterized by PXRD alone or PXRD in combination with one or more of DSC, TGA and ¹H NMR described above.

In some embodiments, crystalline Form B is at least 70%, 80%, 85%, 90%, 95%, 97%, 99%, 99.5% or 99.9% pure. The purity of Form B is determined by dividing the weight of crystalline Form B in a composition comprising compound 1 over the total weight of the compound in the composition. In one embodiment, the present disclosure provides a composition comprising compound 1, wherein at least 70%, 80%, 85%, 90%, 95%, 97%, 99%, 99.5% or 99.9% by weight of the compound in the composition is crystalline Form B of the compound.

In one aspect, the present disclosure provides a method for preparing crystalline Form B of compound 1. In one embodiment, the method is a slurry method described herein. In a particular embodiment of the above disclosed method, crystalline Form B can be obtained using crude compound 1 (e.g., amorphous form or other solid form) as starting material, ethyl acetate as the solvent. In one embodiment, crystalline Form B can be obtained by slurrying crude compound 1 (e.g., amorphous form or other solid form) in ethyl acetate at room temperature (RT) or a temperature that is below RT (e.g., a temperature between 0° C. and 20° C., between 0° C. and 15° C., or between 0° C. and 10° C., or at 5° C., 10° C., 15° C. or 20° C. for a period time (e.g., for 1 hour to 8 hours, for 1 hour to 4 hours, for one day to one week, for 2 or more weeks, for 1, 2, 3, 4, 5, 6 or 7 days, etc.) that is sufficient to form Form B. In some embodiment, crystalline Form B can be obtained by slurrying crude compound 1 in ethyl acetate at 10° C.

In another aspect, the present disclosure provides crystalline Form I of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide maleate salt (maleate salt of compound 1).

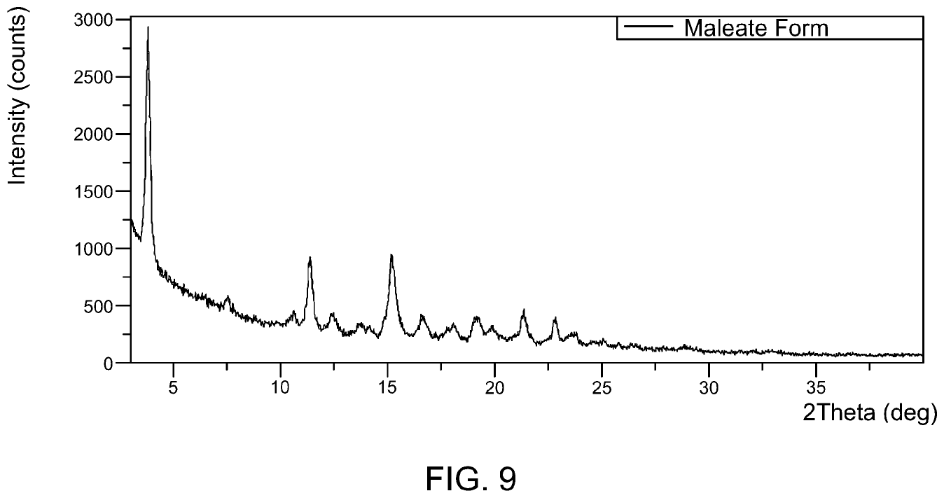

In one embodiment, crystalline Form I is characterized by at least three or at least four powder X-ray diffraction (PXRD) peaks at 2θ angles selected from 3.9°, 11.4°, 15.2°, 16.6° and 19.2°. In another embodiment, crystalline Form I is characterized by PXRD peaks at 2θ angles of 3.9°, 11.4°, 15.2°, 16.6° and 19.2°. In another embodiment, crystalline Form I is characterized by at least three, at least four, at least five, or at least six PXRD peaks at 2θ angles selected from 3.9°, 11.4°, 12.4°, 15.2°, 16.6°, 19.2° and 21.3°. In yet another embodiment, crystalline Form I is characterized by PXRD peaks at 2θ angles of 3.9°, 11.4°, 12.4°, 15.2°, 16.6°, 19.2° and 21.3°. In some embodiments, the peaks described in the above embodiments for crystalline Form I have a relative intensity of at least 1%, at least 2%, at least 5%, at least 10%, or at least 15%. In some embodiments, crystalline Form I has a PXRD pattern that is substantially the same as PXRD pattern shown in FIG. 9.

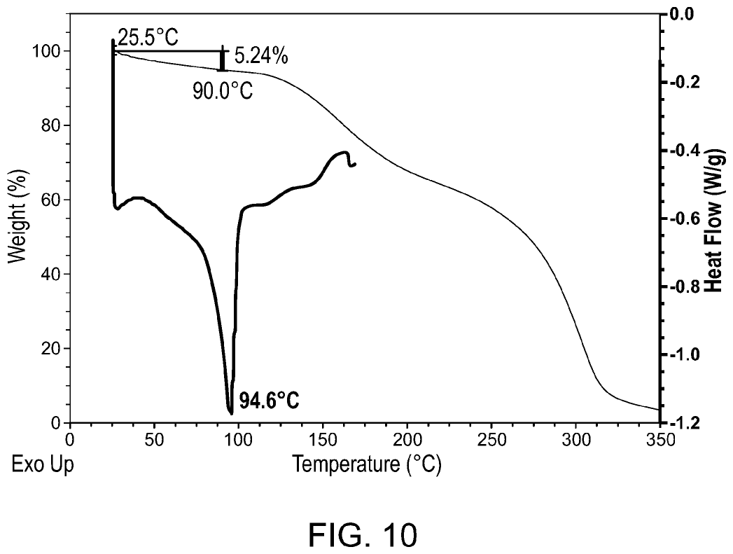

In some embodiments, crystalline Form I has a DSC profile that is substantially the same as DSC profile shown in FIG. 10. In particular, crystalline Form I has a melting temperature of 94.6° C.±2° C.

In some embodiments, crystalline Form I has a TGA profile that is substantially the same as the TGA profile shown in FIG. 10.

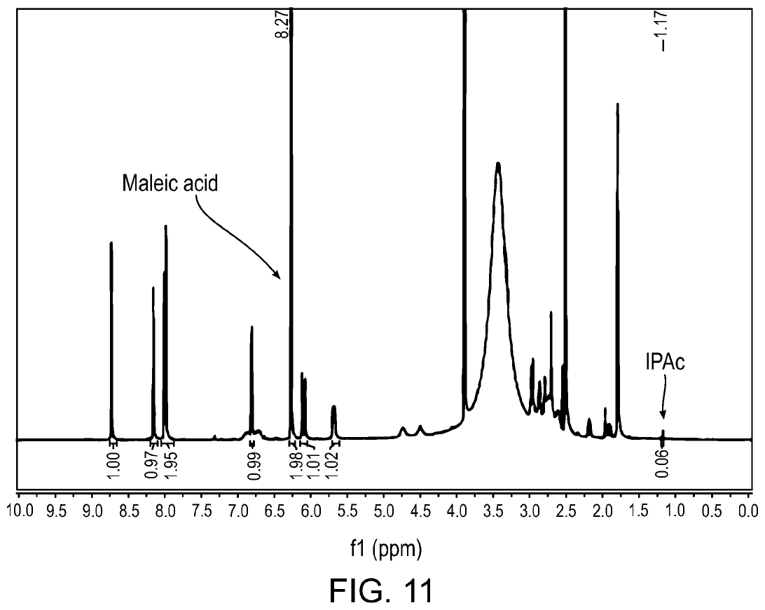

In another embodiment, crystalline Form I is characterized by the ¹H NMR as shown in FIG. 11.

In some embodiments, crystalline Form I is characterized by, for example, ¹H NMR DSC, TGA and PXRD. In one embodiment, crystalline Form I is characterized by PXRD alone or PXRD in combination with one or more of DSC, TGA and ¹H NMR described above.

In some embodiments, crystalline Form I is at least 70%, 80%, 85%, 90%, 95%, 97%, 99%, 99.5% or 99.9% pure. The purity of Form I is determined by dividing the weight of crystalline Form I in a composition comprising maleate salt of compound 1 over the total weight of maleate salt of the compound in the composition. In one embodiment, the present disclosure provides a composition comprising maleate salt of compound 1, wherein at least 70%, 80%, 85%, 90%, 95%, 97%, 99%, 99.5% or 99.9% by weight of maleate salt of the compound in the composition is crystalline Form I.

In one aspect, the present disclosure provides a method for preparing crystalline Form I of maleate salt of compound 1. In one embodiment, the method is a slurry method described herein. In a particular embodiment of the above disclosed method, crystalline Form I can be obtained using Form B and maleic acid as starting materials and isopropyl acetate (IPAC) as the solvent. In certain embodiments, Form B of compound 1 and maleic acid are mixed in isopropyl acetate at room temperature to yield a clear solution. The solution then is cooled to a low temperature to form a solid followed by slurrying at the low temperature for a period of time (e.g., for 1 hour to 8 hours, for 1 hour to 4 hours, for one day to one week, for 2 or more weeks, for 1, 2, 3, 4, 5, 6 or 7 days, etc.) sufficient to form crystalline Form I of the maleate salt of compound 1. In some embodiments, 0.5 to 1.5, 0.8 to 1.2 or 0.9 to 1.1 molar equivalent of maleic acid relative to compound 1 can be used. In some embodiments, the solution is cooled to a temperature between −30° C. and 0° C., between −30° C. and −5° C., between −30° C. and −10° C. or between −25° C. and −15° C. In some embodiments, the solution is cooled to −20° C. In some embodiments, crystalline Form I can be obtained by dissolving Form B of compound 1 and maleic acid in isopropyl acetate at RT to form a clear solution, followed by cooling the solution to −20° C. to form a solid and slurrying the solid at −20° C. for 1 day.

In another aspect, the present disclosure provides crystalline Form II of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide tartrate salt (tartrate salt of compound 1).

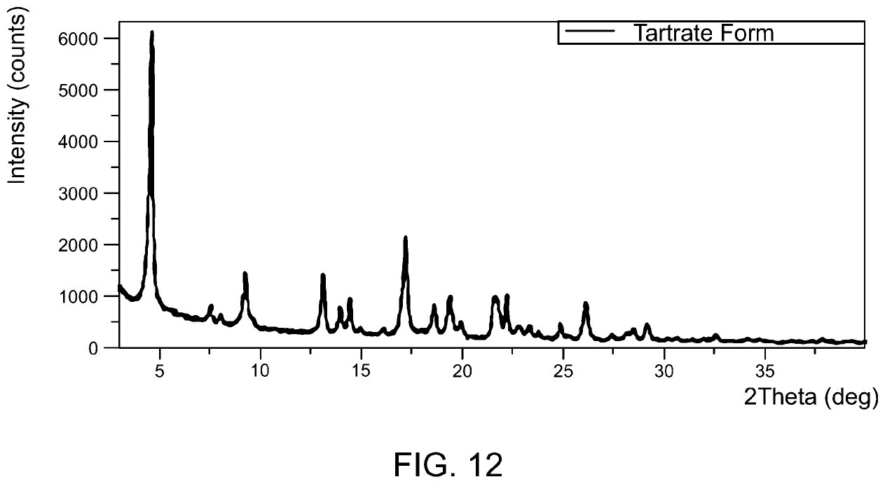

In one embodiment, crystalline Form II is characterized by at least three or at least four powder X-ray diffraction (PXRD) peaks at 2θ angles selected from 4.6°, 9.2°, 13.10, 17.2° and 21.7°. In another embodiment, crystalline Form II is characterized by PXRD peaks at 2θ angles of 4.6°, 9.2°, 13.10, 17.2° and 21.7°. In another embodiment, crystalline Form II is characterized by at least three, at least four, at least five, at least six, at least seven, at least eight, at least nine or at least ten PXRD peaks at 2θ angles selected from 4.6°, 9.2°, 13.10, 14.0°, 14.4°, 17.2°, 18.6°, 19.4°, 21.7°, 22.1° and 26.1°. In yet another embodiment, crystalline Form II is characterized by PXRD peaks at 2θ angles of 4.6°, 9.2°, 13.1°, 14.0°, 14.4°, 17.2°, 18.6°, 19.4°, 21.7°, 22.1° and 26.1°. In some embodiments, the peaks described in the above embodiments for crystalline Form II have a relative intensity of at least 1%, at least 2%, at least 5%, at least 10%, or at least 15%. In some embodiments, crystalline Form II has a PXRD pattern that is substantially the same as PXRD pattern shown in FIG. 12.

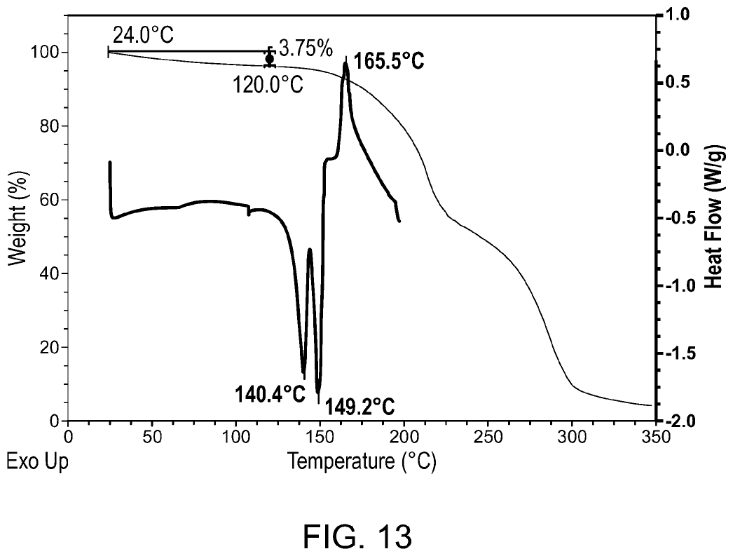

In some embodiments, crystalline Form II has a DSC profile that is substantially the same as DSC profile shown in FIG. 13. In particular, crystalline Form II is characterized by endothermic peaks of 140.4° C.±2° C. and 149.2° C.±2° C. and an exothermic peak at 165.5° C.±2° C.

In some embodiments, crystalline Form II has a TGA profile that is substantially the same as the TGA profile shown in FIG. 13.

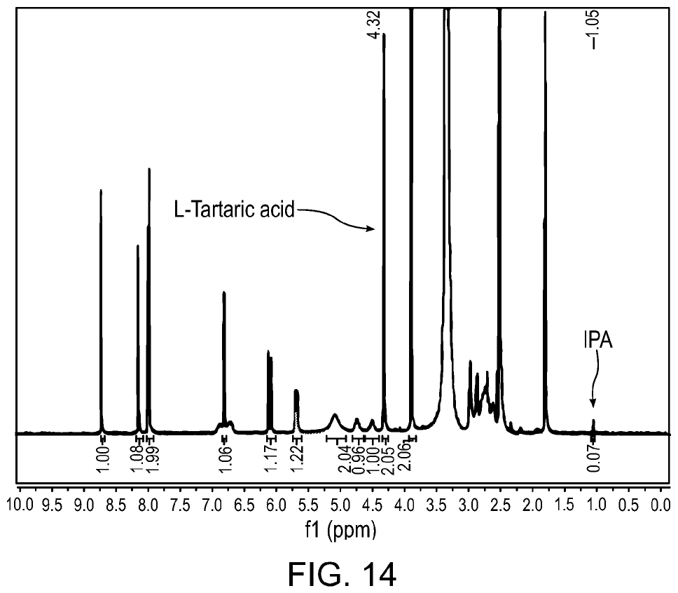

In another embodiment, crystalline Form II is characterized by the ¹H NMR as shown in FIG. 14.

In some embodiments, crystalline Form II is characterized by, for example, ¹H NMR DSC, TGA and PXRD. In one embodiment, crystalline Form II is characterized by PXRD alone or PXRD in combination with one or more of DSC, TGA and ¹H NMR described above.

In some embodiments, crystalline Form II is at least 70%, 80%, 85%, 90%, 95%, 97%, 99%, 99.5% or 99.9% pure. The purity of Form II is determined by dividing the weight of crystalline Form II in a composition comprising tartrate salt of compound 1 over the total weight of the tartrate salt of the compound in the composition. In one embodiment, the present disclosure provides a composition comprising tartrate salt of compound 1, wherein at least 70%, 80%, 85%, 90%, 95%, 97%, 99%, 99.5% or 99.9% by weight of the tartrate salt of the compound in the composition is crystalline Form II.

In one aspect, the present disclosure provides a method for preparing crystalline Form II of N-methyl-N-((1s,3s)-3-methyl-3-((6-(1-methyl-H-pyrazol-4-yl)pyrazolo[1,5-a]pyrazin-4-yl)oxy)cyclobutyl)acrylamide tartrate salt. In one embodiment, the method is a slurry method described herein. In a particular embodiment of the above disclosed method, crystalline Form II can be obtained using crystalline Form B as starting material, with L-tartaric acid, and IPA as the solvent. In certain embodiments, Form B of compound 1 and tartaric acid are mixed in IPA at room temperature to yield a clear solution. The solution then is cooled to a low temperature to form a solid followed by slurrying at the low temperature for a period of time (e.g., for 1 hour to 8 hours, for 1 hour to 4 hours, for one day to one week, for 2 or more weeks, for 1, 2, 3, 4, 5, 6 or 7 days, etc.) sufficient to form crystalline Form II of the tartrate salt of compound 1. In some embodiments, 0.5 to 1.5, 0.8 to 1.2 or 0.9 to 1.1 molar equivalent of tartaric acid relative to compound 1 can be used. In some embodiments, the solution is cooled to a temperature between −20° C. and 10° C., between −10° C. and 10° C., between 0° C. and 10° C., or between 0° C. and 5° C. In some embodiments, the solution is cooled to 5° C. In some embodiments, crystalline Form II can be obtained by dissolving Form B of compound 1 and tartaric acid (1:1 molar equivalent) in IPA at room temperature to form a clear solution, followed by cooling the solution to 5° C. to form a solid and slurrying the solid at 5° C. for 1 day. In some embodiments, the tartaric acid and tartrate described above is L-tartaric acid and L-tartrate respectively.

In another aspect, the present disclosure provides crystalline Form III of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide tartrate salt (tartrate salt of compound 1).

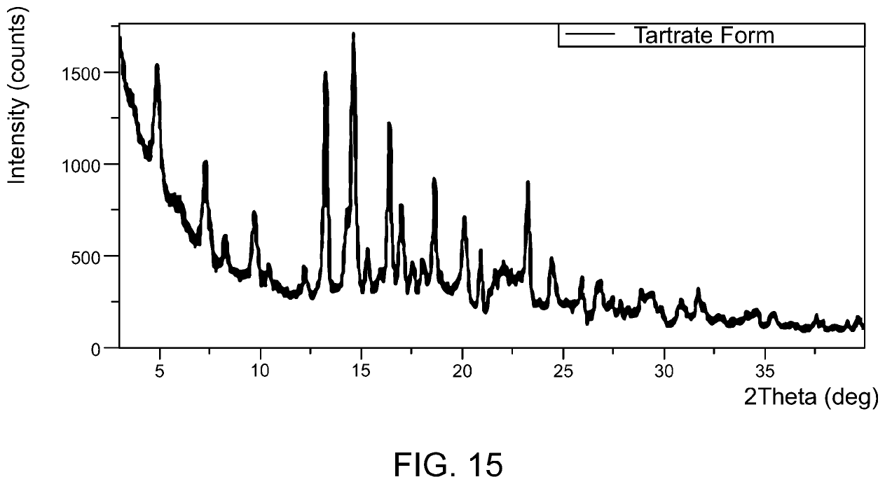

In one embodiment, crystalline Form III is characterized by at least three, at least four, at least five, at least six or at least seven powder X-ray diffraction (PXRD) peaks at 2θ angles selected from 4.9°, 7.3°, 9.7°, 13.2°, 14.6°, 16.4°, 18.6° and 23.3°. In another embodiment, crystalline Form III is characterized by PXRD peaks at 2θ angles of 4.9°, 7.3°, 9.7°, 13.2°, 14.6°, 16.4°, 18.6° and 23.3°. In another embodiment, crystalline Form III is characterized by at least three, at least four, at least five, at least six, at least seven, at least eight, at least nine, at least ten, at least eleven, at least twelve or at least thirteen PXRD peaks at 2θ angles selected from 4.9°, 7.3°, 9.7° 13.2°, 14.4°, 14.6°, 15.3°, 16.4°, 17.0°, 18.6°, 20.1°, 20.9°, 23.3° and 24.5°. In yet another embodiment, crystalline Form III is characterized by PXRD peaks at 2θ angles of 4.9°, 7.3°, 9.7° 13.2°, 14.4°, 14.6°, 15.3°, 16.4°, 17.0°, 18.6°, 20.1°, 20.9°, 23.3° and 24.5°. In some embodiments, the peaks described in the above embodiments for crystalline Form III have a relative intensity of at least 1%, at least 2%, at least 5%, at least 10%, or at least 15%. In some embodiments, crystalline Form III has a PXRD pattern that is substantially the same as PXRD pattern shown in FIG. 15.

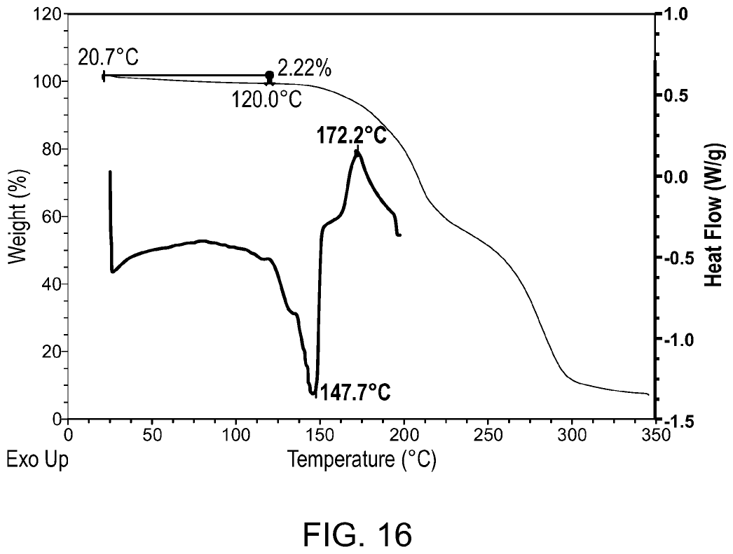

In some embodiments, crystalline Form III has a DSC profile that is substantially the same as DSC profile shown in FIG. 16. In particular, crystalline Form III is characterized by a melting temperature of 147.7° C.±2° C. and an exothermic peak at 172.2° C.±2° C. in the DSC profile.

In some embodiments, crystalline Form III has a TGA profile that is substantially the same as the TGA profile shown in FIG. 16.

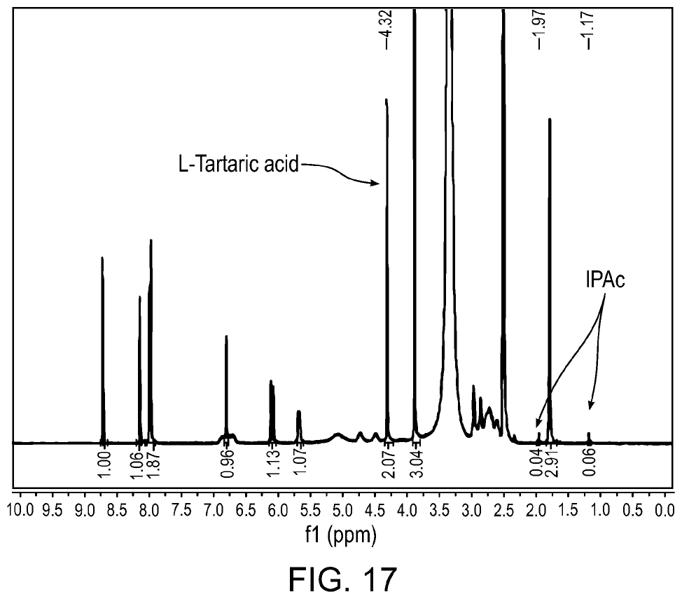

In another embodiment, crystalline Form III is characterized by the ¹H NMR as shown in FIG. 17.

In some embodiments, crystalline Form III is characterized by, for example, ¹H NMR DSC, TGA and PXRD. In one embodiment, crystalline Form III is characterized by PXRD alone or PXRD in combination with one or more of DSC, TGA and ¹H NMR described above.

In some embodiments, crystalline Form III is at least 70%, 80%, 85%, 90%, 95%, 97%, 99%, 99.5% or 99.9% pure. The purity of Form III is determined by dividing the weight of crystalline Form III in a composition comprising compound 1 tartrate salt over the total weight of the tartrate salt of compound 1 in the composition. In one embodiment, the present disclosure provides a composition comprising tartrate salt of compound 1, wherein at least 70%, 80%, 85%, 90%, 95%, 97%, 99%, 99.5% or 99.9% by weight of the tartrate salt of compound 1 in the composition is crystalline Form III.

In one aspect, the present disclosure provides a method for preparing crystalline Form III of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide tartrate salt. In one embodiment, the method is a slurry method described herein. In a particular embodiment of the above disclosed method, crystalline Form III can be obtained using crystalline Form B as starting material, with L-tartaric acid, and IPAc as the solvent. In certain embodiments, Form B of compound 1 and tartaric acid are mixed in IPAc and stirred at room temperature for a period of time (e.g., for 1 hour to 8 hours, for 1 hour to 4 hours, for one day to one week, for 2 or more weeks, for 1, 2, 3, 4, 5, 6 or 7 days, etc.) sufficient to form crystalline Form III of the tartrate salt of compound 1. In some embodiments, 0.5 to 1.5, 0.8 to 1.2 or 0.9 to 1.1 molar equivalent of tartaric acid relative to compound 1 can be used. In one embodiment, crystalline Form III can be obtained by stirring crystalline Form B and tartaric acid (1:1 molar equivalent) in IPAc at RT for 1 day. In some embodiments, the tartaric acid and tartrate described above is L-tartaric acid and L-tartrate respectively.

In another aspect, the present disclosure provides crystalline Form IV of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide citrate salt.

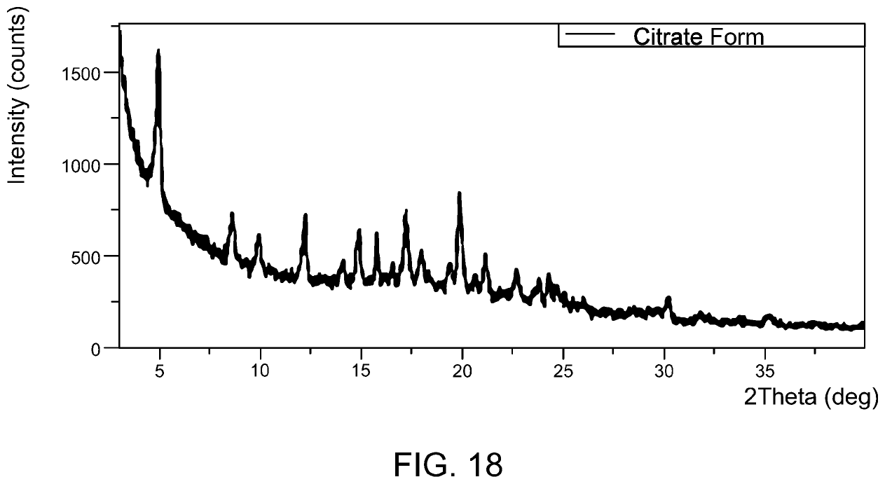

In one embodiment, crystalline Form IV is characterized by at least three, at least four, at least five or at least six powder X-ray diffraction (PXRD) peaks at 2θ angles selected from 4.9°, 8.6°, 9.9°, 12.2°, 14.9°, 18.0° and 19.9°. In another embodiment, crystalline Form IV is characterized by PXRD peaks at 2θ angles of 4.9°, 8.6°, 9.9°, 12.2°, 14.9°, 18.0° and 19.9°. In another embodiment, crystalline Form IV is characterized by at least three, at least four, at least five, at least six, at least seven, at least eight or at least nine PXRD peaks at 2θ angles selected from 4.9°, 8.6°, 9.9°, 12.2°, 14.9°, 15.8°, 18.0°, 19.9°, 21.2° and 22.7°. In yet another embodiment, crystalline Form IV is characterized by PXRD peaks at 2θ angles of 4.9°, 8.6°, 9.9°, 12.2°, 14.9°, 15.8°, 18.0°, 19.9°, 21.2° and 22.7°. In some embodiments, the peaks described in the above embodiments for crystalline Form IV have a relative intensity of at least 1%, at least 2%, at least 5%, at least 10%, or at least 15%. In some embodiments, crystalline Form IV has a PXRD pattern that is substantially the same as PXRD pattern shown in FIG. 18.

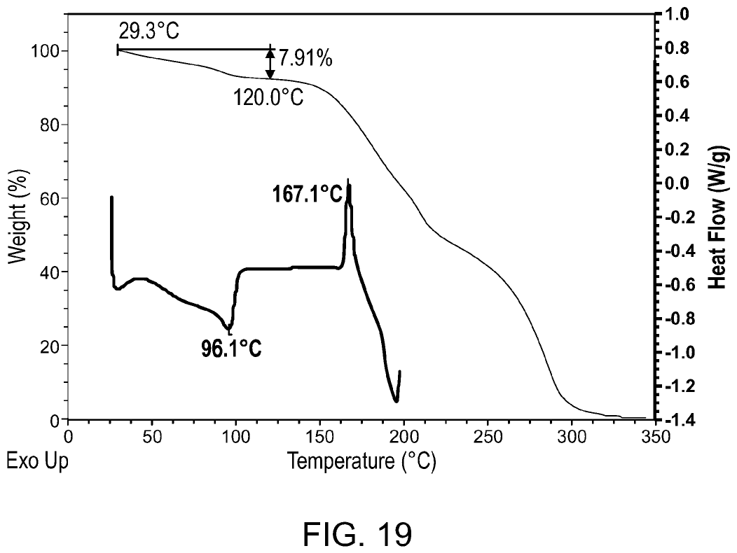

In some embodiments, crystalline Form IV has a DSC profile that is substantially the same as DSC profile shown in FIG. 19. In particular, crystalline Form IV is characterized by a melting temperature of 96.1° C.±2° C. and an exothermic peak at 167.1° C.±2° C.

In some embodiments, crystalline Form IV has a TGA profile that is substantially the same as the TGA profile shown in FIG. 19.

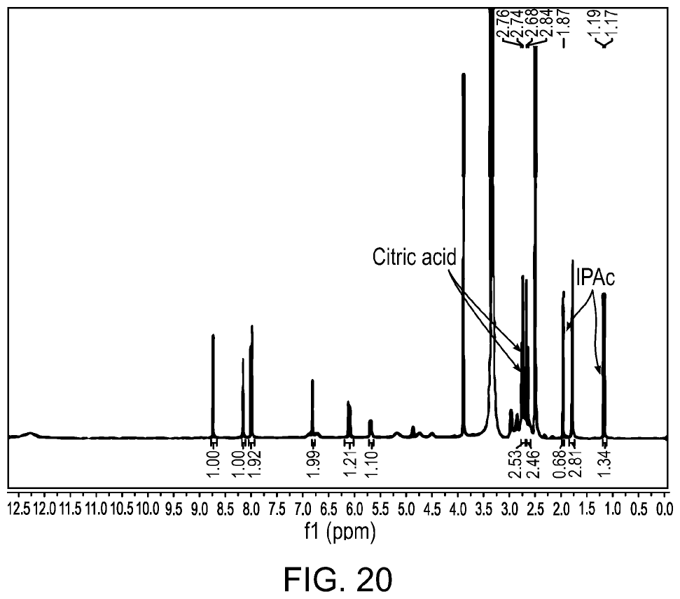

In another embodiment, crystalline Form IV is characterized by the ¹H NMR as shown in FIG. 20.

In some embodiments, crystalline Form IV is characterized by, for example, ¹H NMR DSC, TGA and PXRD. In one embodiment, crystalline Form IV is characterized by PXRD alone or PXRD in combination with one or more of DSC, TGA and ¹H NMR described above.

In some embodiments, crystalline Form IV is at least 70%, 80%, 85%, 90%, 95%, 97%, 99%, 99.5% or 99.9% pure. The purity of Form IV is determined by dividing the weight of crystalline Form IV in a composition comprising citrate salt of compound 1 over the total weight of citrate salt of the compound in the composition. In one embodiment, the present disclosure provides a composition comprising citrate salt of compound 1, wherein at least 70%, 80%, 85%, 90%, 95%, 97%, 99%, 99.5% or 99.9% by weight of citrate salt of the compound in the composition is crystalline Form IV.

In one aspect, the present disclosure provides a method for preparing crystalline Form IV of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide citrate salt. In one embodiment, the method is a slurry method described herein. In a particular embodiment of the above disclosed method, crystalline Form IV can be obtained using crystalline Form B as starting material, with citric acid, and IPAc as the solvent. In certain embodiments, Form B of compound 1 and citric acid are mixed in IPAc and stirred at room temperature for a period of time (e.g., for 1 hour to 8 hours, for 1 hour to 4 hours, for one day to one week, for 2 or more weeks, for 1, 2, 3, 4, 5, 6 or 7 days, etc.) sufficient to form crystalline Form IV of the tartrate salt of compound 1. In some embodiments, 0.5 to 1.5, 0.8 to 1.2 or 0.9 to 1.1 molar equivalent of citric acid relative to compound 1 can be used. In one embodiment, crystalline Form IV can be obtained by stirring crystalline Form B and citric acid (1:1 molar equivalent) in IPAc at RT for 1 day.

In another aspect, the present disclosure provides crystalline Form V of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide proline salt (proline salt of compound 1).

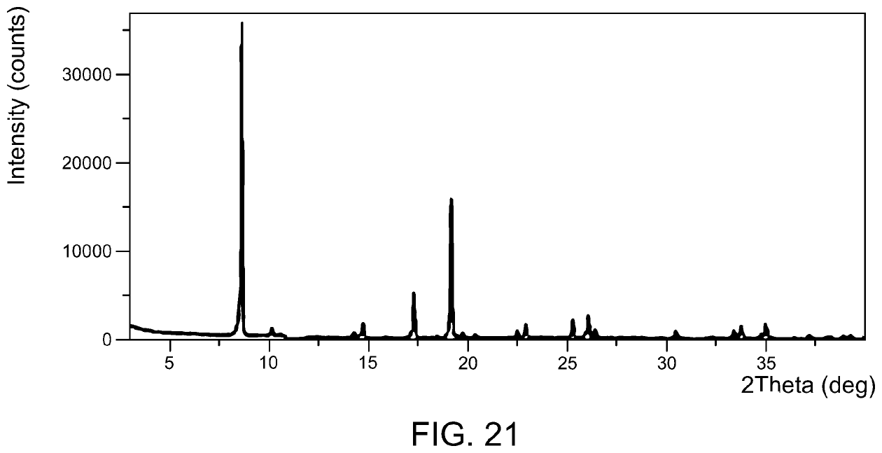

In one embodiment, crystalline Form V is characterized by at least three powder X-ray diffraction (PXRD) peaks at 2θ angles selected from 8.6°, 17.3°, 19.10 and 26.0°. In another embodiment, crystalline Form V is characterized by PXRD peaks at 2θ angles of 8.6°, 17.3°, 19.1° and 26.0°. In another embodiment, crystalline Form V is characterized by at least three, at least four, at least five or at least six PXRD peaks at 2θ angles selected from 8.6°, 14.7°, 17.3°, 19.1°, 22.9°, 25.2° and 26.0°. In yet another embodiment, crystalline Form V is characterized by PXRD peaks at 2θ angles of 8.6°, 14.7°, 17.3°, 19.1°, 22.9°, 25.2° and 26.0°. In some embodiments, the peaks described in the above embodiments for crystalline Form V have a relative intensity of at least 1%, at least 2%, at least 5%, at least 10%, or at least 15%. In some embodiments, crystalline Form V has a PXRD pattern that is substantially the same as PXRD pattern shown in FIG. 21.

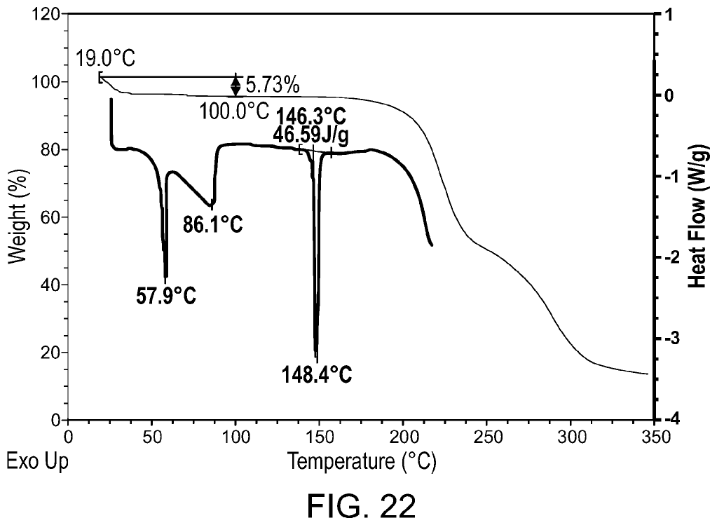

In some embodiments, crystalline Form V has a DSC profile that is substantially the same as DSC profile shown in FIG. 22. In particular, crystalline Form V is characterized by two endothermic peaks at 57.9° C.±2° C., 86.1° C.±2° C., and a melting temperature of 148.4° C.±2° C.

In some embodiments, crystalline Form V has a TGA profile that is substantially the same as the TGA profile shown in FIG. 22.

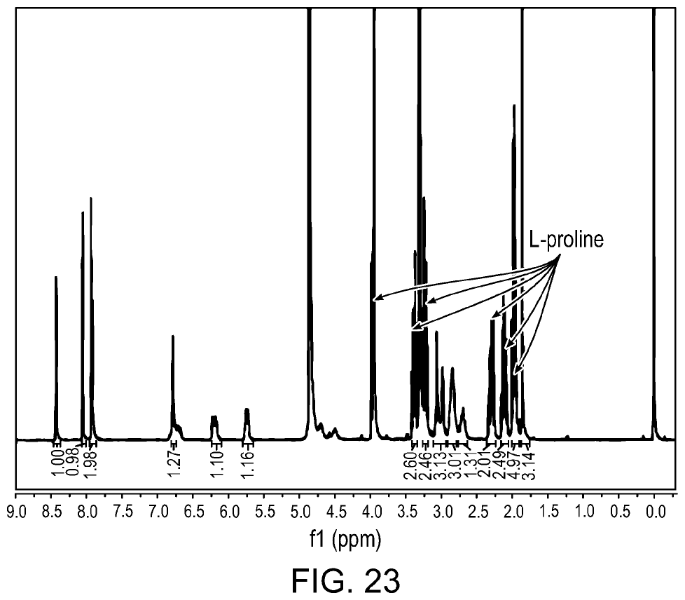

In another embodiment, crystalline Form V is characterized by the ¹H NMR as shown in FIG. 23.

In some embodiments, crystalline Form V is characterized by, for example, ¹H NMR DSC, TGA and PXRD. In one embodiment, crystalline Form V is characterized by PXRD alone or PXRD in combination with one or more of DSC, TGA and ¹H NMR described above.

In some embodiments, crystalline Form V is at least 70%, 80%, 85%, 90%, 95%, 97%, 99%, 99.5% or 99.9% pure. The purity of Form V is determined by dividing the weight of crystalline Form V in a composition comprising proline salt of compound 1 over the total weight of the proline salt of the compound in the composition. In one embodiment, the present disclosure provides a composition comprising proline salt of the compound, wherein at least 70%, 80%, 85%, 90%, 95%, 97%, 99%, 99.5% or 99.9% by weight of the proline salt of compound in the composition is crystalline Form V of the compound.

In one aspect, the present disclosure provides a method for preparing crystalline Form V of N-methyl-N-((1s,3s)-3-methyl-3-((6-(1-methyl-H-pyrazol-4-yl)pyrazolo[1,5-a]pyrazin-4-yl)oxy)cyclobutyl)acrylamide proline salt. In one embodiment, the method is a slurry method described herein. In a particular embodiment of the above disclosed method, crystalline Form V can be obtained using crystalline Form B as starting material, with proline, and IPA as the solvent. In certain embodiments, Form B of compound 1 and proline are mixed in IPA and stirred at room temperature for a period of time (e.g., for 1 hour to 8 hours, for 1 hour to 4 hours, for one day to one week, for 2 or more weeks, for 1, 2, 3, 4, 5, 6 or 7 days, etc.) sufficient to form crystalline Form V of proline salt of compound 1. In some embodiments, 0.5 to 1.5, 0.8 to 1.2 or 0.9 to 1.1 molar equivalent of proline relative to compound 1 can be used. In one embodiment, crystalline Form V can be obtained by stirring crystalline Form B and proline (1:1 molar equivalent) in IPA at RT for 1 day.

It will be understood that the 20 values of the PXRD pattern for crystalline Form A, B, I, II, III, IV or V may vary slightly from one instrument to another and may depend on variations in sample preparation. Therefore, the PXRD peak positions for these crystalline Forms are not to be construed as absolute and can vary ±0.2°.

As intended herein, “substantially the same PXRD pattern as shown in FIG. x” mean that for comparison purposes, at least 80%, at least 90%, or at least 95% of the peaks shown in FIG. x are present. FIG. x is FIG. 1, FIG. 5, FIG. 9, FIG. 12, FIG. 15, FIG. 18 or FIG. 21. It is to be further understood that for comparison purposes some variability in peak position from those shown in FIG. x are allowed, such as ±0.2°. Similarly, for comparison purposes some variability in peak position from those shown in DSC and TGA profiles as well as NMR spectrum described herein are allowed. For example, the peak positions can vary from those shown in FIG. 4, such as ±0.5 ppm. The onset temperature and/or melting temperature can vary from those shown FIG. 3, FIG. 7, FIG. 10, FIG. 13, FIG. 16, FIG. 19 or FIG. 22, such as ±2° C.

In one aspect, the present disclosure provides a pharmaceutical composition comprising a crystalline form described herein, e.g., crystalline Form A, B, I, II, III, IV or V, and a pharmaceutically acceptable excipient.

Another aspect of the disclosure provides a method of treating a disorder responsive to inhibition of Bruton's tyrosine kinase in a subject comprising administering to the subject an effective amount of crystalline Form A, B, I, II, III, IV or V or a composition (e.g., pharmaceutical composition) comprising crystalline Form A, B, I, II, III, IV or V.

In some embodiments of the above disclosed aspect, the disorder is an autoimmune disorder. In some additional embodiments of the above disclosed aspect, the autoimmune disorder is multiple sclerosis.

The term “autoimmune disorders” includes diseases or disorders involving inappropriate immune response against native antigens, such as acute disseminated encephalomyelitis (ADEM), Addison's disease, alopecia areata, antiphospholipid antibody syndrome (APS), autoimmune hemolytic anemia, autoimmune hepatitis, bullous pemphigoid (BP), Coeliac disease, dermatomyositis, diabetes mellitus type 1, Goodpasture's syndrome, Graves' disease, Guillain-Barre syndrome (GBS), Hashimoto's disease, idiopathic thrombocytopenic purpura, lupus erythematosus, mixed connective tissue disease, multiple sclerosis, myasthenia gravis, pemphigus vulgaris, pernicious anaemia, polymyositis, primary biliary cirrhosis, Sjogren's syndrome, temporal arteritis, and Wegener's granulomatosis. The term “inflammatory disorders” includes diseases or disorders involving acute or chronic inflammation such as allergies, asthma, prostatitis, glomerulonephritis, pelvic inflammatory disease (PID), inflammatory bowel disease (IBD, e.g., Crohn's disease, ulcerative colitis), reperfusion injury, rheumatoid arthritis, transplant rejection, and vasculitis. In some embodiments, the present disclosure provides a method of treating rheumatoid arthritis or lupus. In some embodiments, the present disclosure provides a method of treating multiple sclerosis.

The term “cancer” includes diseases or disorders involving abnormal cell growth and/or proliferation, such as glioma, thyroid carcinoma, breast carcinoma, lung cancer (e.g. small-cell lung carcinoma, non-small-cell lung carcinoma), gastric carcinoma, gastrointestinal stromal tumors, pancreatic carcinoma, bile duct carcinoma, ovarian carcinoma, endometrial carcinoma, prostate carcinoma, renal cell carcinoma, lymphoma (e.g., anaplastic large-cell lymphoma), leukemia (e.g. acute myeloid leukemia, T-cell leukemia, chronic lymphocytic leukemia), multiple myeloma, malignant mesothelioma, malignant melanoma, and colon cancer (e.g. microsatellite instability-high colorectal cancer). In some embodiments, the present disclosure provides a method of treating leukemia or lymphoma.

As used herein, the term “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the term “treating” or “treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

The effective dose of a compound provided herein, or a pharmaceutically acceptable salt thereof, administered to a subject can be 10 μg-500 mg.

Administering a compound described herein to a mammal comprises any suitable delivery method. Administering a compound described herein to a mammal includes administering a compound described herein topically, enterally, parenterally, transdermally, transmucosally, via inhalation, intracisternally, epidurally, intravaginally, intravenously, intramuscularly, subcutaneously, intradermally or intravitreally to the mammal. Administering a compound described herein to a mammal also includes administering topically, enterally, parenterally, transdermally, transmucosally, via inhalation, intracisternally, epidurally, intravaginally, intravenously, intramuscularly, subcutaneously, intradermally or intravitreally to a mammal a compound that metabolizes within or on a surface of the body of the mammal to a compound described herein.

Thus, a compound described herein, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that an effective dosage level will be obtained.

The tablets, troches, pills, capsules, and the like can include the following: binders such as gum tragacanth, acacia, corn starch or gelatin; excipients such as dicalcium phosphate; a disintegrating agent such as corn starch, potato starch, alginic acid and the like; a lubricant such as magnesium stearate; or a sweetening agent such as sucrose, fructose, lactose or aspartame or a flavoring agent.

The active compound may also be administered intravenously or intraperitoneally by infusion or injection. Solutions of the active compound or its salts can be prepared in water, optionally mixed with a nontoxic surfactant.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols or glycols or water-alcohol/glycol blends, in which the compounds described herein can be dissolved or dispersed at effective levels, optionally with the aid of non-toxic surfactants.

Useful dosages of a compound described herein can be determined by comparing their in vitro activity, and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound described herein, required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.1 to about 10 mg/kg of body weight per day.

The compound described herein can be conveniently administered in unit dosage form; for example, containing 0.01 to 10 mg, or 0.05 to 1 mg, of active ingredient per unit dosage form. In some embodiments, a dose of 5 mg/kg or less can be suitable.

The desired dose may conveniently be presented in a single dose or as divided doses administered at appropriate intervals.

The disclosed method can include a kit comprising a compound described herein and instructional material which can describe administering a compound described herein or a composition comprising a compound described herein to a cell or a subject. This should be construed to include other embodiments of kits that are known to those skilled in the art, such as a kit comprising a (such as sterile) solvent for dissolving or suspending a compound described herein or composition prior to administering a compound described herein or composition to a cell or a subject. In some embodiments, the subject can be a human.

### EXEMPLIFICATIONS

### Powder X-Ray Diffraction

Crystallinity of the compound was studied using a XRD-D8 X-ray powder diffractometer using Cu Ka radiation (Bruker, Madison, WI). The instrument is equipped with a long fine focus X-ray tube. The tube voltage and amperage were set to 40 kV and 40 mA, respectively. The divergence and scattering slits were set at 1° and the receiving slit was set at 0.15 mm. Diffracted radiation was detected by a Lynxeye detector. A θ-2θ continuous scan at 1.6°/min from 3 to 42° 2θ was used. The sample was prepared for analysis by placing it on a zero background plate.

### Differential Scanning Calorimetry (DSC) and Thermogravimetric Analysis (TGA)

Thermal properties of the compound were examined using a Discovery Differential Scanning Calorimeter (DSC) (TA Instruments) and a Discovery Thermogravimetric Analyzer (TGA) (TA Instruments). Sample was enclosed in a closed aluminum DSC pan for DSC analysis and in an open aluminum pan for TGA analysis. The thermal analysis was performed with a linear gradient from 25° C. to 300° C. at 10° C. per minute for both DSC and TGA studies.

### Single Crystal X-Ray Diffraction

A clean crystal was selected and mounted with LV Cryo-oil™ on a plastic loop. A specimen (dimensions 0.157 mm×0.095 mm×0.070 mm) was used for the X-ray crystallographic analysis. The X-ray intensity data were measured at a wavelength of λ=1.54178 Å at 100K with a Rigaku Synergy-S single crystal diffractometer with a HyPix-6000HE Hybrid Photon Counting detector. The structure was solved and refined using the OLEX2 Package with SHELX XS and XL program.

### Synthesis of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide (Compound 1)

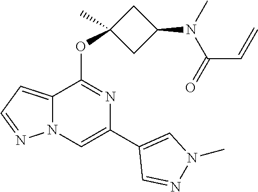 [SMILES: C=CC(=O)N(C)[C@H]1C[C@@](C)(Oc2nc(-c3cnn(C)c3)cn3nccc23)C1]

### Synthesis of tert-butyl methyl((1s,3s)-3-methyl-3-((6-(1-methyl-1H-pyrazol-4-yl)pyrazolo[1,5-a]pyrazin-4-yl)oxy)cyclobutyl)carbamate

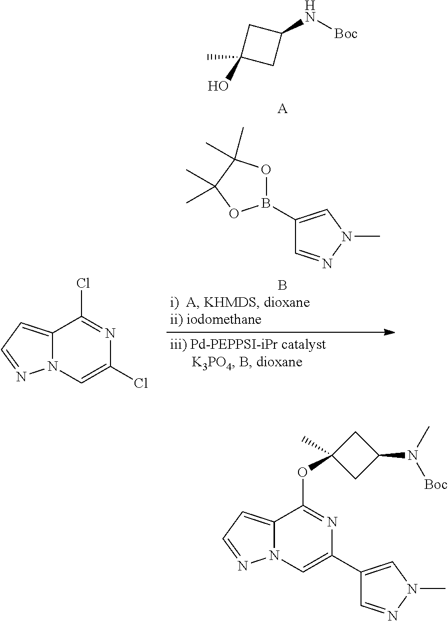

In a 100-mL one-necked round bottom flask equipped with a condenser under a nitrogen atmosphere, potassium hexamethyldisilazide (1 M in THF, 2.2 mL) was added to a solution of tert-butyl ((1s,3s)-3-hydroxy-3-methylcyclobutyl)carbamate (150 mg, 0.75 mmol) in dioxane (7.5 mL) at room temperature. After 5 min, a solution of 4,6-dichloropyrazolo[1,5-a]pyrazine (128 mg, 0.68 mmol) in dioxane (2.5 mL) was dropwise added to the thick white suspension. Iodomethane (240 mg, 1.70 mmol, 105 μL) was added to the resulting orange suspension at room temperature and stirring was continued for an additional 30 min. The resulting reaction mixture was degassed by purging with nitrogen for 30 min, after which a degassed solution of potassium phosphate tribasic (531 mg, 2.50 mmol) in water (2.5 mL) was added at RT. After an additional 10 min of purging of the clear orange reaction mixture with nitrogen, a previously degassed solution of 1-methyl-4-(4,4,5,5-tetramethyl-1,3,2-dioxaborolan-2-yl)pyrazole (212 mg, 1.02 mmol) in dioxane (2.0 mL) was added followed by solid Pd-PEPPSI™-IPr catalyst (93 mg, 0.14 mmol). After purging of the reaction mixture with nitrogen for additional 15 min, the reaction mixture was heated at reflux for 3 hours. To the vigorously stirred reaction mixture were ethyl acetate (20 mL) followed by water (20 mL). After 30 min, the organic phase was separated, and the volatiles were removed under reduced pressure. The resulting residue was purified by column chromatography (40 g silica gel, 0-80% [3:1 EtOAc:EtOH] with 2% NH₄OH modifier in heptanes) yielding the title compound as a pale yellow oil (130 mg, 47% yield). LCMS m/z=413.1 (M+H)+. ¹H NMR (500 MHz, methanol-d₄) δ ppm 8.42 (s, 1H), 8.05 (s, 1H), 7.93 (s, 1H), 7.91 (d, J=2.44 Hz, 1H), 6.77 (d, J=1.22 Hz, 1H), 4.10-4.45 (m, 1H), 3.95 (s, 3H), 2.82 (s, 3H), 2.74-2.81 (m, 2H), 2.67 (br s, 2H), 1.81 (s, 3H), 1.46 (s, 9H).

### Synthesis of (1s,3s)-N,3-dimethyl-3-((6-(1-methyl-1H-pyrazol-4-yl)pyrazolo[1,5-a]pyrazin-4-yl)oxy)cyclobutan-1-amine

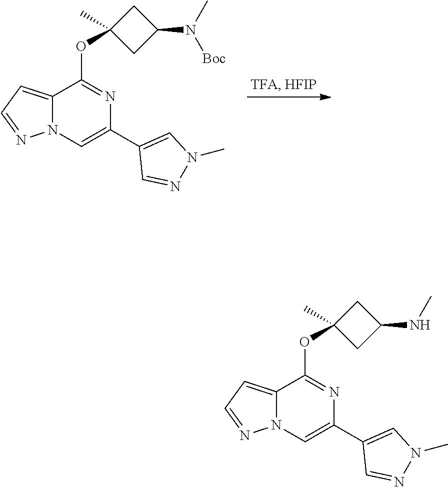 [SMILES: Cn1cc(-c2cn3nccc3c(O[C@]3(C)C[C@H](N(C)C(=O)OC(C)(C)C)C3)n2)cn1 ; CN[C@H]1C[C@@](C)(Oc2nc(-c3cnn(C)c3)cn3nccc23)C1]

To a solution of tert-butyl methyl((1s,3s)-3-methyl-3-((6-(1-methyl-1H-pyrazol-4-yl)pyrazolo[1,5-a]pyrazin-4-yl)oxy)cyclobutyl)carbamate (4.05 g, 9.82 mmol) in HFIP (45 mL) was added TFA (2.24 g, 19.6 mmol, 1.5 mL) at room temperature. The resulting reaction mixture was stirred overnight. Ethyl acetate (50 mL) was added followed by a sat. aq. NaHCO₃ solution (25 mL) and brine (10 mL). After vigorous stirring for 30 min, the organic phase was separated, dried over sodium sulfate, filtered, and concentrated. The resulting residue was purified by column chromatography (24 g silica gel, 80-100% [3:1 EtOAc:EtOH] with 2% NH₄OH modifier in heptanes) yielding the title compound as a pale yellow gum (2.53 g, 82% yield). LCMS m/z=313.1 (M+H)+. ¹H NMR (500 MHz, methanol-d₄) δ ppm 8.41 (d, J=1.22 Hz, 1H), 8.05 (s, 1H), 7.86-7.97 (m, 2H), 6.72-6.81 (m, 1H), 3.95 (s, 3H), 2.96-3.11 (m, 1H), 2.76-2.90 (m, 2H), 2.31 (s, 3H), 2.25-2.31 (m, 2H), 2.25-2.31 (m, 2H), 1.80 (s, 3H).

### Synthesis of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide

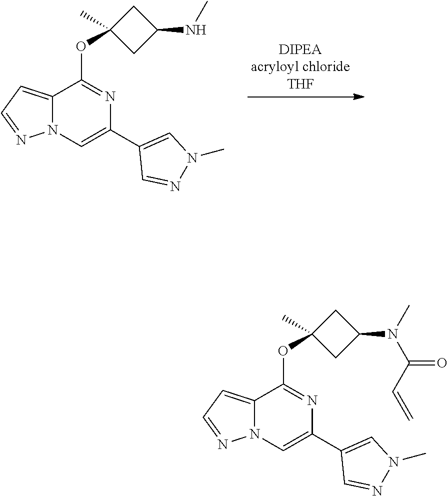 [SMILES: CN[C@H]1C[C@@](C)(Oc2nc(-c3cnn(C)c3)cn3nccc23)C1 ; C=CC(=O)N(C)[C@H]1C[C@@](C)(Oc2nc(-c3cnn(C)c3)cn3nccc23)C1]

To a solution of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide (4.05 g, 9.82 mmol) and DIPEA (2.81 g, 21.7 mmol, 3.8 mL) in THF (50 mL) was added acryloyl chloride (819 mg, 9.04 mmol, 740 μL) at 0° C. After 30 min, the reaction mixture was diluted with EtOAc (50 mL) and a sat. aq. NaHCO₃ solution (50 mL) were added. The vigorously stirred biphasic mixture was brought to room temperature and stirring was continued for another 30 min. The organic phase was separated, washed with water (25 mL) and brine (25 mL), dried over Na₂SO₄, filtered, and concentrated. The resulting residue was purified by column chromatography (80 g silica gel, 0-100% [3:1 EtOAc:EtOH] with 2% NH₄OH modifier in heptanes). The colorless solid was recrystallized from EtOAc/heptanes (1/3, 45 mL) to afford the title compound as a free flowing crystalline solid (1.8 g, 68% yield). Melting point=137.5° C. LCMS m/z=389.1.1 (M+Na)+. ¹H NMR (500 MHz, methanol-d₄) δ ppm 8.44 (s, 1H), 8.06 (s, 1H), 7.85-7.98 (m, 2H), 6.67-6.85 (m, 2H), 6.12-6.26 (m, 1H), 5.74 (br d, J=9.16 Hz, 1H), 4.45-4.77 (m, 1H), 3.95 (s, 3H), 2.94-3.12 (m, 3H), 2.62-2.94 (m, 4H), 1.86 (s, 3H).

### Example 1. Preparation of Crystalline Form A

Form A was obtained by slurrying Form B in IPA at RT for 6 days. Crystalline form A was analyzed using PXRD, TGA and DSC. The PXRD pattern of crystalline Form A is shown in FIG. 1 and the main peaks are listed in Table 1A. A weight loss of 0.6% up to 150° C. was observed on TGA curve as shown in FIG. 3. DSC profile exhibiting one endothermic peak at 137.3° C. (onset temperature) is shown in FIG. 3. ¹H NMR result (FIG. 4) showed no IPA was detected. Single crystal structure is shown in FIG. 2.

TABLE 1A
PXRD peak list for crystalline Form A

| | Net | Relative |
|---|---|---|
| 2θ angle | Intensity | Intensity (%) |
| 8.5 | 82.1 | 6.2 |
| 10.1 | 176 | 13.3 |
| 10.6 | 1322 | 100.0 |
| 11.9 | 145 | 11.0 |
| 14.3 | 965 | 73.0 |
| 16.9 | 718 | 54.3 |
| 17.8 | 629 | 47.6 |
| 18.2 | 549 | 41.5 |
| 19.4 | 593 | 44.9 |
| 21.3 | 65.1 | 4.9 |
| 22.2 | 250 | 18.9 |
| 23.0 | 165 | 12.5 |
| 24.0 | 80.8 | 6.1 |
| 25.1 | 1069 | 80.9 |
| 27.2 | 92.7 | 7.0 |
| 27.8 | 269 | 20.3 |
| 28.7 | 92.7 | 7.0 |
| 30.7 | 117 | 8.9 |
| 34.9 | 76.2 | 5.8 |
| 36.1 | 87.5 | 6.6 |

TABLE 1B
Single crystal data and structure refinement for Form A

| | |
|---|---|
| Empirical formula | C₁₉H₂₂N₆O₂ |
| Formula weight | 366.42 |
| Temperature/K | 100.01(11) |
| Crystal system | triclinic |
| Space group | P-1 |
| a/Å | 5.98064(4) |
| b/Å | 10.28273(10) |
| c/Å | 14.91842(10) |
| α/° | 93.5318(6) |
| β/° | 97.7016(6) |
| γ/° | 93.7913(7) |
| Volume/Å³ | 904.872(12) |
| Z | 2 |
| ρ_{calc}g/cm³ | 1.345 |
| μ/mm⁻¹ | 0.745 |
| F(000) | 388.0 |
| Crystal size/mm³ | 0.157 × 0.095 × 0.07 |
| Radiation | Cu Kα (λ = 1.54184) |

Negligible weight loss in TGA and neat a DSC profile were observed, indicating Form A is an anhydrate.

### Example 2. Preparation of Crystalline Form B

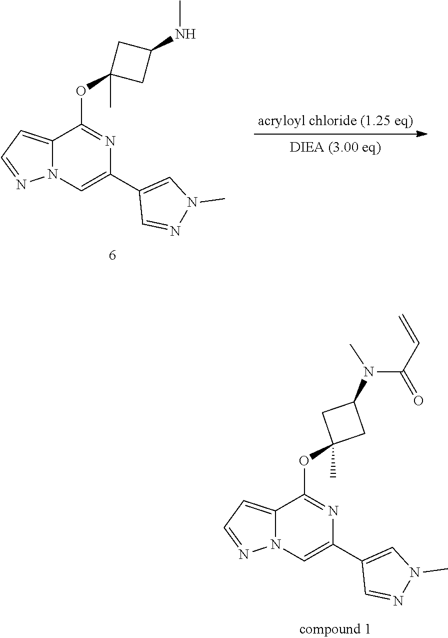 [SMILES: CN[C@H]1C[C@@](C)(Oc2nc(-c3cnn(C)c3)cn3nccc23)C1 ; C=CC(=O)N(C)[C@H]1C[C@@](C)(Oc2nc(-c3cnn(C)c3)cn3nccc23)C1]

THF (2.53 L), and compound 6 (230 g, 1.00 eq) were added to a 5 L reactor at 15-20° C. To the solution at 15-20° C., was added DIEA (285 g, 3.00 eq) over 10 mins. The mixture was cooled to 0° C. for 5 minutes. Acroloyl chloride (83.3 g, 1.25 eq) was added in to the mixture at 0-5° C. The mixture was stirred at 0° C. for 1 hr. Upon completion of the reaction (monitored by HPLC), the reaction was quenched with slow addition of water (1150 mL). The organic layer was separated and the aqueous layer was extracted with MTBE (3×920 mL). The combined organic layer was washed with 0.2 M HCl (575 mL), dried over Na₂SO₄, filtered and concentrated. The crude material was slurried with EtOAc (1150 mL) at 10° C. to afford crystalline form B (99.6% purity, 97.0% QNMR) as a white solid. The PXRD pattern of crystalline Form B is shown in FIG. 5 and the main peaks are listed in Table 2A. A weight loss of 0.5% up to 150° C. was observed on TGA curve as shown in FIG. 7. DSC profile exhibiting one endothermic peak at (147.3° C., onset temp) is shown in FIG. 7. ¹H NMR (FIG. 8) showed no residual solvent was detected. Single crystal structure is shown in FIG. 6.

TABLE 2A
PXRD peak list for crystalline Form B

| | Net | Relative |
|---|---|---|
| 2θ angle | Intensity | Intensity (%) |
| 8.6 | 558 | 12.3 |
| 9.3 | 256 | 5.7 |
| 10.2 | 4522 | 100.0 |
| 12.7 | 143 | 3.2 |
| 13.2 | 89.6 | 2.0 |
| 14.4 | 1085 | 24.0 |
| 16.0 | 3020 | 66.8 |
| 17.3 | 4408 | 97.5 |
| 18.7 | 388 | 8.6 |
| 20.4 | 2857 | 63.2 |
| 21.4 | 424 | 9.4 |
| 21.9 | 857 | 19.0 |
| 22.7 | 1459 | 32.3 |
| 25.4 | 4370 | 96.6 |
| 26.4 | 287 | 6.3 |
| 27.8 | 104 | 2.3 |
| 28.4 | 202 | 4.5 |
| 30.7 | 267 | 5.9 |
| 32.1 | 192 | 4.2 |
| 37.6 | 294 | 6.5 |

TABLE 2B
Single crystal data and structure refinement for Form B

| | |
|---|---|
| Empirical formula | C₁₉H₂₂N₆O₂ |
| Formula weight | 366.42 |
| Temperature/K | 100.01(11) |
| Crystal system | triclinic |
| Space group | P-1 |
| a/Å | 5.98064(4) |
| b/Å | 10.28273(10) |
| c/Å | 14.91842(10) |
| α/° | 93.5318(6) |
| β/° | 97.7016(6) |
| γ/° | 93.7913(7) |
| Volume/Å³ | 904.872(12) |
| Z | 2 |
| ρ_{calc}g/cm³ | 1.345 |
| μ/mm⁻¹ | 0.745 |
| F(000) | 388.0 |
| Crystal size/mm³ | 0.157 × 0.095 × 0.07 |
| Radiation | Cu Kα (λ = 1.54184) |

Negligible weight loss in TGA and neat a DSC profile were observed, indicating Form B is an anhydrate.

### Example 3. Preparation of Crystalline Form I

Equimolar quantities of Form B and maleic acid were slurried in IPAc at room temperature to afford a clear solution. The mixture was cooled at −20° C. for one day to afford Form I as solid. The solids were vacuum-dried at RT before characterization. The PXRD pattern of crystalline Form I is shown in FIG. 9 and the main peaks are listed in Table 3. ¹H NMR shown in FIG. 11 indicates that molar ratio of maleic acid to N-methyl-N-((1s,3s)-3-methyl-3-((6-(1-methyl-1H-pyrazol-4-yl)pyrazolo[1,5-a]pyrazin-4-yl)oxy)cyclobutyl)acrylamide is 1.0 and that of the residual solvent IPAc to N-methyl-N-((1s,3s)-3-methyl-3-((6-(1-methyl-1H-pyrazol-4-yl)pyrazolo[1,5-a]pyrazin-4-yl)oxy)cyclobutyl)acrylamide is 0.01 (0.2 wt %). A weight loss of 5.2% up to 90° C. was observed on TGA curve as shown in FIG. 10. The DSC analysis shows that Form I has a melting temperature of 94.6° C. (FIG. 10).

TABLE 3
PXRD peak list for crystalline Form I

| | Net | Relative |
|---|---|---|
| 2θ angle | Intensity | Intensity (%) |
| 3.9 | 1841 | 100.0 |
| 7.4 | 85.9 | 4.7 |
| 10.5 | 55.6 | 3.0 |
| 11.4 | 598 | 32.5 |
| 12.4 | 132 | 7.2 |
| 13.8 | 76.8 | 4.2 |
| 15.2 | 655 | 35.6 |
| 16.6 | 162 | 8.8 |
| 18.0 | 90.3 | 4.9 |
| 19.2 | 187 | 10.2 |
| 19.8 | 78.4 | 4.3 |
| 21.3 | 278 | 6.1 |
| 22.8 | 206 | 4.6 |
| 23.6 | 87.9 | 1.9 |

### Example 4. Preparation of Crystalline Form II

Equimolar quantities of Form B and L-tartaric acid were slurried in IPA at room temperature to afford a clear solution. The mixture was cooled at 5° C. for one day to afford Form II as solid. The solids were vacuum-dried at RT before characterization. The PXRD pattern of crystalline Form II is shown in FIG. 12 and the main peaks are listed in Table 4. ¹H NMR shown in FIG. 14 indicates that molar ratio of L-tartaric acid to N-methyl-N-((1s,3s)-3-methyl-3-((6-(1-methyl-1H-pyrazol-4-yl)pyrazolo[1,5-a]pyrazin-4-yl)oxy)cyclobutyl)acrylamide is 1.0 and that of the residual solvent IPA to N-methyl-N-((1s,3s)-3-methyl-3-((6-(1-methyl-1H-pyrazol-4-yl)pyrazolo[1,5-a]pyrazin-4-yl)oxy)cyclobutyl)acrylamide is 0.01 (0.2 wt %). A weight loss of 3.8% up to 120° C. was observed on TGA curve as shown in FIG. 13. The DSC analysis shows that Form II has two endothermic signals at 140. 4° C., 149.2° C. (peak) and one exothermic signal at 165.5° C. (FIG. 13).

TABLE 4
PXRD peak list for crystalline Form II

| | Net | Relative |
|---|---|---|
| 2θ angle | Intensity | Intensity (%) |
| 4.6 | 4392 | 100.0 |
| 7.5 | 217 | 4.9 |
| 8.0 | 106 | 2.4 |
| 9.2 | 1026 | 23.4 |
| 13.1 | 1020 | 23.2 |
| 14.0 | 395 | 9.0 |
| 14.4 | 509 | 11.6 |
| 17.2 | 1767 | 40.2 |
| 18.6 | 546 | 12.4 |
| 19.4 | 742 | 16.9 |
| 19.9 | 213 | 4.8 |
| 21.7 | 781 | 17.8 |
| 22.1 | 397 | 9.0 |
| 24.9 | 265 | 6.0 |
| 26.1 | 708 | 16.1 |
| 29.1 | 273 | 6.2 |

### Example 5. Preparation of Crystalline Form III

Equimolar quantities of Form B and L-tartaric acid were slurried in IPAc at room temperature to afford Form III as a solid. The solids were vacuum-dried at RT before characterization. The PXRD pattern of crystalline Form III is shown in FIG. 15 and the main peaks are listed in Table 5. ¹H NMR shown in FIG. 17 indicates that molar ratio of L-tartaric acid to N-methyl-N-((1s,3s)-3-methyl-3-((6-(1-methyl-1H-pyrazol-4-yl)pyrazolo[1,5-a]pyrazin-4-yl)oxy)cyclobutyl)acrylamide is 1.0 and that of the residual solvent IPAc to N-methyl-N-((1s,3s)-3-methyl-3-((6-(1-methyl-1H-pyrazol-4-yl)pyrazolo[1,5-a]pyrazin-4-yl)oxy)cyclobutyl)acrylamide is 0.01 (0.2 wt %). A weight loss of 2.2% up to 120° C. was observed on TGA curve as shown in FIG. 16. The DSC analysis shows that Form III has one endothermic signal at 147.7° C. (peak) and one exothermic signal at 172.2° C. (FIG. 16).

TABLE 5
PXRD peak list for crystalline Form III

| | Net | Relative |
|---|---|---|
| 2θ angle | Intensity | Intensity (%) |
| 4.9 | 580 | 42.7 |
| 7.3 | 478 | 35.2 |
| 8.3 | 165 | 12.2 |
| 9.7 | 362 | 26.7 |
| 13.2 | 1191 | 87.8 |
| 14.4 | 360 | 26.5 |
| 14.6 | 1357 | 100.0 |
| 15.3 | 205 | 15.1 |
| 16.4 | 893 | 65.8 |
| 17.0 | 455 | 33.5 |
| 18.6 | 627 | 46.2 |
| 20.1 | 438 | 32.3 |
| 20.9 | 275 | 20.3 |
| 23.3 | 632 | 46.6 |
| 24.5 | 254 | 18.7 |

### Example 6. Preparation of Crystalline Form IV

Equimolar quantities of Form B and citric acid were slurried in IPAc at room temperature to afford Form IV as a solid. The solids were vacuum-dried at RT before characterization. The PXRD pattern of crystalline Form IV is shown in FIG. 18 and the main peaks are listed in Table 6. ¹H NMR shown in FIG. 20 indicates that molar ratio of citric acid to N-methyl-N-((1s,3s)-3-methyl-3-((6-(1-methyl-1H-pyrazol-4-yl)pyrazolo[1,5-a]pyrazin-4-yl)oxy)cyclobutyl)acrylamide is 1.22 (partially overlapped) and that of the residual solvent IPAc to N-methyl-N-((1s,3s)-3-methyl-3-((6-(1-methyl-1H-pyrazol-4-yl)pyrazolo[1,5-a]pyrazin-4-yl)oxy)cyclobutyl)acrylamide is 0.22 (3.9 wt %). A weight loss of 7.9% up to 120° C. was observed on TGA curve as shown in FIG. 19. The DSC analysis shows that Form IV has one endothermic signal at 96.1° C. (peak) and one exothermic signal at 167.1° C. (FIG. 19).

TABLE 6
PXRD peak list for crystalline Form IV

| | Net | Relative |
|---|---|---|
| 2θ angle | Intensity | Intensity (%) |
| 4.9 | 637 | 100.0 |
| 8.6 | 218 | 34.2 |
| 9.9 | 165 | 25.9 |
| 12.2 | 235 | 36.9 |
| 14.1 | 86.1 | 13.5 |
| 14.9 | 219 | 34.4 |
| 15.8 | 186 | 29.2 |
| 16.5 | 81.7 | 12.8 |
| 18.0 | 329 | 51.6 |
| 18.9 | 119 | 18.7 |
| 19.9 | 445 | 69.9 |
| 21.2 | 148 | 23.2 |
| 22.7 | 144 | 22.6 |
| 24.3 | 133 | 20.9 |
| 30.2 | 104 | 16.3 |

### Example 7. Preparation of Crystalline Form V

Equimolar quantities of Form B and L-proline were slurried in IPA at room temperature to afford Form V as a solid. The solids were vacuum-dried at RT before characterization. The PXRD pattern of crystalline Form V is shown in FIG. 21 and the main peaks are listed in Table 7. ¹H NMR shown in FIG. 23 indicates that molar ratio of L-proline to N-methyl-N-((1s,3s)-3-methyl-3-((6-(1-methyl-1H-pyrazol-4-yl)pyrazolo[1,5-a]pyrazin-4-yl)oxy)cyclobutyl)acrylamide is 2.5 and no residual solvent IPA was observed. A weight loss of 5.7% up to 100° C. was observed on TGA curve as shown in FIG. 22. The DSC analysis shows that Form V has three endothermic signals at 57.9° C., 86.1° C., 148.4° C. (peak) (FIG. 22).

TABLE 6
PXRD peak list for crystalline Form V

| | Net | Relative |
|---|---|---|
| 2θ angle | Intensity | Intensity (%) |
| 8.6 | 20164 | 100.0 |
| 10.1 | 655 | 3.2 |
| 14.2 | 463 | 2.3 |
| 14.7 | 1369 | 6.8 |
| 17.3 | 4093 | 20.3 |
| 19.1 | 14863 | 73.7 |
| 22.5 | 724 | 3.6 |
| 22.9 | 1611 | 8.0 |
| 25.2 | 1840 | 9.1 |
| 26.0 | 2160 | 10.7 |
| 26.4 | 971 | 4.8 |
| 30.4 | 768 | 3.8 |
| 33.6 | 793 | 3.9 |
| 33.7 | 1215 | 6.0 |
| 35.0 | 1572 | 7.8 |

1. Crystalline Form A of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide.

2. The crystalline Form A of claim 1, wherein the crystalline form is characterized by at least three, at least four, at least five, at least six or at least seven powder X-ray diffraction (PXRD) peaks at 2θ angles selected from 10.1°, 10.6°, 14.3°, 16.9°, 17.8°, 18.2°, 19.4° and 25.1°.

3. The crystalline Form A of claim 1, wherein the crystalline form is characterized by PXRD peaks at 2θ angles of 10.1°, 10.6°, 14.3°, 16.9°, 17.8°, 18.2°, 19.4° and 25.1°.

4. The crystalline Form A of any one of claims 1-3, wherein the crystalline form is characterized by PXRD peaks at 2θ angles selected from 10.1°, 10.6°, 11.9°, 14.3°, 16.9°, 17.8°, 18.2°, 19.4°, 21.3°, 22.2°, 23.0°, 24.0°, 25.1° and 27.8°.

5. The crystalline Form A of any one of claims 1-4, wherein the crystalline Form A is characterized by a melting temperature of 138.4° C.±2° C. determined by differential scanning calorimetry (DSC) analysis.

6. The crystalline Form A of any one of claims 1-5, wherein the crystalline Form A is a non-hygroscopic anhydrate.

7. The crystalline Form A of claim 1, wherein the crystalline form is characterized as a space group P-1 space group.

8. The crystalline Form A of claim 1, wherein the crystalline form is characterized by an asymmetric unit cell with a volume of 904.872 Å3 and 3-D parameters of a=5.98064 Å; b=10.28273 Å; c=14.91842 Å.

9. The crystalline Form A of any one of claims 1-8, wherein the crystalline Form A is at least 70%, 80%, 85%, 90%, 95%, 97%, 99%, 99.5% or 99.9% pure.

10. Crystalline Form B of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide.

11. The crystalline Form B of claim 10, wherein the crystalline form is characterized by at least three, at least four, at least five, at least six or at least seven powder X-ray diffraction (PXRD) peaks at 2θ angles selected from 8.6°, 9.3°, 10.2°, 14.4°, 16.0°, 17.3°, 20.4°, and 25.4°.

12. The crystalline Form B of claim 10, wherein the crystalline form is characterized by PXRD peaks at 2θ angles of 8.6°, 9.3°, 10.2°, 14.4°, 16.0°, 17.3°, 20.4°, and 25.4°.

13. The crystalline Form B of any one of claims 10-12, wherein the crystalline form is characterized by PXRD peaks at 2θ angles selected from 8.6°, 9.3°, 10.2°, 14.4°, 16.0°, 17.3°, 18.7°, 20.4°, 21.4°, 21.9°, 22.7°, 25.4° and 28.4°.

14. The crystalline Form B of any one of claims 10-13, wherein the crystalline Form B is characterized by a melting temperature of 148.3° C.±2° C. determined by differential scanning calorimetry (DSC) analysis.

15. The crystalline Form B of any one of claims 10-14, wherein the crystalline Form B is a non-hygroscopic anhydrate.

16. The crystalline Form B of claim 10, wherein the crystalline form is characterized as a space group P21/c space group.

17. The crystalline Form A of claim 10, wherein the crystalline form is characterized by an asymmetric unit cell with a volume of 1859.45 Å3 and 3-D parameters of a=9.4633 Å; b=20.6615 Å; c=9.5408 Å.

18. The crystalline Form B of any one of claims 10-17, wherein the crystalline Form B is at least 70%, 80%, 85%, 90%, 95%, 97%, 99%, 99.5% or 99.9% pure.

19. Crystalline Form I of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide maleate salt.

20. The crystalline Form I of claim 19, wherein the crystalline form is characterized by at least three or at least four powder X-ray diffraction (PXRD) peaks at 2θ angles selected from 3.9°, 11.4°, 15.2°, 16.6° and 19.2°.

21. The crystalline Form I of claim 19, wherein the crystalline form is characterized by PXRD peaks at 2θ angles of 3.9°, 11.4°, 15.2°, 16.6° and 19.2°.

22. The crystalline Form I of any one of claims 19-21, wherein the crystalline form is characterized by PXRD peaks at 2θ angles selected from 3.9°, 11.4°, 12.4°, 15.2°, 16.6°, 19.2° and 21.3°.

23. The crystalline Form I of any one of claims 19-22, wherein the crystalline Form I is characterized by a melting temperature of 94.6° C.±2° C. determined by differential scanning calorimetry (DSC) analysis.

24. The crystalline Form I of any one of claims 19-23, wherein the crystalline Form I is at least 70%, 80%, 85%, 90%, 95%, 97%, 99%, 99.5% or 99.9% pure.

25. Crystalline Form II of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide tartrate salt.

26. The crystalline Form II of claim 25, wherein the crystalline form is characterized by at least three or at least four powder X-ray diffraction (PXRD) peaks at 2θ angles selected from 4.6°, 9.2°, 13.1°, 17.2° and 21.7°.

27. The crystalline Form II of claim 25, wherein the crystalline form is characterized by PXRD peaks at 2θ angles of 4.6°, 9.2°, 13.1°, 17.2° and 21.7°.

28. The crystalline Form II of any one of claims 25-27, wherein the crystalline form is characterized by PXRD peaks at 2θ angles selected from 4.6°, 9.2°, 13.1°, 14.0°, 14.4°, 17.2°, 18.6°, 19.4°, 21.7°, 22.1° and 26.1°.

29. The crystalline Form II of any one of claims 25-28, wherein the crystalline Form II is characterized by endothermic peaks of 140.4° C.±2° C., and 149.2° C.±2° C. determined by differential scanning calorimetry (DSC) analysis.

30. The crystalline Form II of any one of claims 25-29, wherein the crystalline Form II is at least 70%, 80%, 85%, 90%, 95%, 97%, 99%, 99.5% or 99.9% pure.

31. Crystalline Form III of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide tartrate salt.

32. The crystalline Form III of claim 31, wherein the crystalline form is characterized by at least three, at least four, at least five, at least six or at least seven powder X-ray diffraction (PXRD) peaks at 2θ angles selected from 4.9°, 7.3°, 9.7°, 13.2°, 14.6°, 16.4°, 18.6° and 23.3°.

33. The crystalline Form III of claim 31, wherein the crystalline form is characterized by PXRD peaks at 2θ angles of 4.9°, 7.3°, 9.7°, 13.2°, 14.6°, 16.4°, 18.6° and 23.3°.

34. The crystalline Form III of any one of claims 31-33, wherein the crystalline form is characterized by PXRD peaks at 2θ angles selected from 4.9°, 7.3°, 9.7°, 13.2°, 14.4°, 14.6°, 15.3°, 16.4°, 17.0°, 18.6°, 20.1°, 20.9°, 23.3° and 24.5°.

35. The crystalline Form III of any one of claims 31-34, wherein the crystalline Form III is characterized by a melting temperature of 147.7° C.±2° C. determined by differential scanning calorimetry (DSC) analysis.

36. The crystalline Form III of any one of claims 31-35, wherein the crystalline Form III is at least 70%, 80%, 85%, 90%, 95%, 97%, 99%, 99.5% or 99.9% pure.

37. Crystalline Form IV of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide citrate salt.

38. The crystalline Form IV of claim 37, wherein the crystalline form is characterized by at least three, at least four, at least five or at least six powder X-ray diffraction (PXRD) peaks at 2θ angles selected from 4.9°, 8.6°, 9.9°, 12.2°, 14.9°, 18.0° and 19.9°.

39. The crystalline Form IV of claim 37, wherein the crystalline form is characterized by PXRD peaks at 2θ angles of 4.9°, 8.6°, 9.9°, 12.2°, 14.9°, 18.0° and 19.9°.

40. The crystalline Form IV of any one of claims 37-39, wherein the crystalline form is characterized by PXRD peaks at 2θ angles selected from 4.9°, 8.6°, 9.9°, 12.2°, 14.9°, 15.8°, 18.0°, 19.9°, 21.2° and 22.7°.

41. The crystalline Form IV of any one of claims 37-40, wherein the crystalline Form IV is characterized by a melting temperature of 96.1° C.±2° C. determined by differential scanning calorimetry (DSC) analysis.

42. The crystalline Form IV of any one of claims 37-41, wherein the crystalline Form IV is at least 70%, 80%, 85%, 90%, 95%, 97%, 99%, 99.5% or 99.9% pure.

43. Crystalline Form V of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide proline salt.

44. The crystalline Form V of claim 43, wherein the crystalline form is characterized by at least three powder X-ray diffraction (PXRD) peaks at 2θ angles selected from 8.6°, 17.3°, 19.1° and 26.0°.

45. The crystalline Form V of claim 43, wherein the crystalline form is characterized by PXRD peaks at 2θ angles of 8.6°, 17.3°, 19.1° and 26.0°.

46. The crystalline Form V of any one of claims 43-45, wherein the crystalline form is characterized by PXRD peaks at 2θ angles selected from 8.6°, 14.7°, 17.3°, 19.1°, 22.9°, 25.2° and 26.0°.

47. The crystalline Form V of any one of claims 43-46, wherein the crystalline Form V is characterized by a melting temperature of 148.4° C.±2° C. determined by differential scanning calorimetry (DSC) analysis.

48. The crystalline Form V of any one of claims 43-47, wherein the crystalline Form V is at least 70%, 80%, 85%, 90%, 95%, 97%, 99%, 99.5% or 99.9% pure.

49. A pharmaceutical composition comprising a pharmaceutically acceptable excipient and (i) a crystalline Form A of any one of claims 1-9, or (ii) a crystalline Form B of any one of claims 10-18, or (iii) a crystalline Form I of any one of claims 19-24, or (iv) a crystalline Form II of any one of claims 25-30, or (v) a crystalline Form III of any one of claims 31-36, or (vi) a crystalline Form IV of any one of claims 37-42, or (vii) a crystalline Form V of any one of claims 43-48.

50. A method of treating a disorder responsive to inhibition of Bruton's tyrosine kinase in a subject comprising administering to the subject an effective amount of (i) a crystalline Form A of any one of claims 1-9, or (ii) a crystalline Form B of any one of claims 10-18, or (iii) a crystalline Form I of any one of claims 19-24, or (iv) a crystalline Form II of any one of claims 25-30, or (v) a crystalline Form III of any one of claims 31-36, or (vi) a crystalline Form IV of any one of claims 37-42, or (vii) a crystalline Form V of any one of claims 43-48.

51. The method of claim 50, wherein the disorder is an autoimmune disorder.

52. The method of claim 51, wherein the autoimmune disorder is multiple sclerosis.
